(19) United States
(12) Patent Application Publication
(10) Pub. No.: US 2023/0407224 A1
(43) Pub. Date: Dec. 21, 2023

(54) A MULTI-SCAFFOLD SYSTEM FOR LARGE SCALE CULTIVATION OF CELLS

(71) Applicant: ALEPH FARMS LTD., Rehovot (IL)

(72) Inventors: Neta Lavon, Ness Ziona (IL); Sagit Shalel Levanon, Modiin (IL); Nadav Tal, Shekef (IL)

(21) Appl. No.: 18/251,222

(22) PCT Filed: Nov. 3, 2021
(86) PCT No.: PCT/IL2021/051302

§ 371 Date: Apr. 28, 2023

Related U.S. Application Data
(60) Provisional application No. 63/109,382, filed on Nov. 4, 2020.

(51) Int. Cl. C12M 1/12; C12M 1/00; C12M 3/00; C12M 1/34; A23L 13/00

(52) U.S. Cl. CPC C12M 25/14; C12M 23/40; C12M 21/08; C12M 41/44; C12M 41/26; C12M 41/34; C12M 41/12; A23L 13/00

(57) ABSTRACT

The present invention is in the field of large-scale production of cultured cells, providing systems comprising a plurality of scaffolds arranged optionally in a multi-layer configuration and methods of use thereof for production of cells and/or tissue cultures for a variety of uses, including the production of cultured food products, particularly cultured meat.

### FIELD OF THE INVENTION

The present invention is in the field of large-scale production of cultured cells, providing systems comprising a plurality of scaffolds and methods of use thereof for production of cells and/or tissue cultures for a variety of uses, including the production of cultured food products, particularly cultured meat.

### BACKGROUND OF THE INVENTION

The approach of combining cells with a biomaterial that acts as a scaffold for tissue development has been taken in a variety of fields, from tissue engineering and repair to the production of cultured food. For all uses, the cells should be capable of propagating on the scaffold and acquiring the required organization and function to produce the desired tissue. The propagated cells and/or the tissue formed may be separated from the scaffold for further use or the entire composition of the scaffold(s) with the propagated cells and/or tissue can be used. In the cultured food industry, scaffolds made of edible materials may contribute to the texture and mouth feel of the cultured food product.

A challenge in the production of cultured cells and/or tissues, aiming at final products with or without scaffolds, remains the scale-up of the systems and processes while keeping the desired characteristics of the cultured cells and/or tissues. The cultured food industry faces even greater challenges as the production must remain cost-effective in view of the price the public would be willing to pay for the end products.

International (PCT) Application Publication No. WO 2019/016795 to the Applicant of the present invention discloses a method for producing an edible composition, comprising incubating a three-dimensional porous scaffold and a plurality of cell types comprising: myoblasts or progenitor cells thereof, at least one type of extracellular matrix (ECM)-secreting cells and potentially endothelial cells or progenitor cells thereof, and inducing myoblasts differentiation into myotubes, as well as edible compositions so produced.

The Applicant of the present invention further disclosed a cultivation system for producing cultured food products, particularly cultured meat, on a commercial scale. The cultivation system comprises at least one cell culture bioreactor, typically a plurality of cell culture bioreactors, for growing non-human-animal-derived adherent cells on at least one scaffold placed within the cell culture bioreactor, a bioreactor made of a flexible bag being a particular example (International (PCT) Application Publication No. WO 2020/222239).

International (PCT) Application Publication No. WO 2019/051486 discloses a bioreactor having tissue scaffolds and having culture medium perfused therethrough. There may be multiple independent culture chambers and reservoirs or sub-reservoirs. Sensors can provide for individually controlling conditions in various culture chambers, and various culture chambers can be operated differently or for different durations. It is possible to infer the number of cells or the progress toward confluence from the fluid resistance of the scaffold, based on flowrate and pressure drop. Harvesting may include any combination or sequence of; exposure to harvesting reagent; vibration; liquid flow that is steady, pulsatile or oscillating; passage of gas-liquid interface through the scaffold. Vibration and flow can be applied so as to reinforce each other.

International (PCT) Application Publication No. WO 2021/102375 discloses apparatuses and systems for preparing a cell-based meat product, and methods of using thereof. Generally, the apparatuses, systems, and methods grow a meat product on one or more enclosed substrates. For example, an apparatus for preparing a meat product may comprise an enclosure comprising a cavity, and a substrate arranged within the cavity and comprising a plurality of nested surfaces curved around a longitudinal axis and a surface configured to support growth of the meat product. The substrate may be configured to receive a fluid substantially parallel to the longitudinal axis.

There is a great need for a variety of systems and methods that can answer the requirements of large-scale production of cultured cells and/or tissues at a cost-effective manner.

### SUMMARY OF THE INVENTION

The present invention answers the above-described needs, providing systems, methods, and bioreactors for advanced cell and/or tissue cultivation, optionally for the production of cultured food products.

According to a first aspect, there is provided a cultivation system for cell and/or tissue cultivation comprising: at least one cell culture bioreactor, the cell culture bioreactor comprising: a first surface comprising an inlet port, a second surface comprising an outlet port, and at least one bioreactor wall, wherein the at least one bioreactor wall is extending from the first surface towards the second surface and is defining an internal chamber configured to accommodate therein at least one scaffold unit; and an inlet manifold fluidly coupled to the inlet port and an outlet manifold fluidly coupled to the outlet port, wherein each manifold comprises openings positioned therealong and extending therethrough configured to direct a flow of a liquid therethrough.

The cultivation system further comprises at least one scaffold unit comprising a plurality of scaffolds, wherein each scaffold is separated from its neighboring or consecutive scaffolds to form spaces therebetween enabling liquid flow, wherein at least part of each scaffold comprises cells, wherein the at least one scaffold unit is configured to be disposed within the at least one cell culture bioreactor.

According to some embodiments, the cultivation system further comprises at least one pump, wherein the manifolds are fluidly coupled to the at least one pump, and wherein the at least one pump is configured to control the flow rate of liquid flowing through the at least one scaffold unit and to circulate the liquid within the system.

According to some embodiments, each scaffold of the plurality of scaffolds is separated from its neighboring or consecutive scaffolds to form spaces, wherein said spaces are configured to enable liquid flow therethrough, between neighboring or consecutive scaffolds of the scaffold unit. In further embodiments, the spaces between neighboring or consecutive scaffolds are configured to enable fluid communication between the inlet manifold and the outlet manifold.

According to some embodiments, each of the scaffolds of the plurality of scaffolds comprises at least two surfaces facing opposite directions along and/or in parallel to a longitudinal axis of said scaffold. According to further embodiments, none of the surfaces defines an enclosed perimeter across any cross-section thereof.

According to some embodiments, each of the scaffolds is planar.

According to some embodiments, the thickness of each of the scaffolds of the plurality of scaffolds ranges from about 1 mm to about 10 cm. According to further embodiments, the thickness of each of the scaffolds ranges from about 1 cm to about 5 cm. According to further embodiments, the thickness of each of the scaffolds ranges from about 1 mm to about 6 mm. According to these embodiments, the dimension of each of the width axis and the length axis of each of the scaffold is at least 10 time higher compared to the thickness of said scaffold.

According to some embodiments, the area of each of the at least two surfaces of each scaffold of the plurality of scaffolds is at least 5 cm². According to further embodiments, the area of each of the two surfaces ranges from about 10 cm² to about 15,000 cm².

According to some embodiments, upon operation of the at least one pump, the liquid flows along the longitudinal axis of each scaffold of the plurality of scaffolds. According to some embodiments, upon operation of the at least one pump, the liquid flows on at least one surface of the scaffold, within the spaces between neighboring scaffolds, or a combination thereof.

According to some embodiments, the scaffolds are made from a porous material. According to further embodiments, the porous material comprises pores having a diameter ranging from about 2 μm to about 1.5 mm. According to still further embodiments, the pores have an average diameter ranging from about 5 μm to about 250 μm. According to still further embodiments, the pores have an average diameter ranging from about 10 μm to about 100 μm. According to some embodiments, a part of pores is interconnected to form channels.

According to some embodiments, each of the scaffolds in the plurality of scaffolds is of the same type.

According to some embodiments, the spaces separating between neighboring scaffolds are even.

According to some embodiments, a height of each space formed between neighboring scaffolds is below about 30 mm. According to further embodiments, the height of each space between neighboring scaffolds ranges from 0.1 mm to 10 mm.

According to some embodiments, the cell culture bioreactor further comprises a plurality of supportive elements separating each scaffold from its neighboring scaffolds. According to further embodiments, each scaffold of the plurality of scaffolds is at least partially placed onto at least one supportive element. According to further embodiments, the plurality of scaffolds is vertically stacked, one on top of the other, along and/or in parallel to a vertical axis, wherein each scaffold is spaced from the following scaffold by at least one supportive element. According to still further embodiments, at least one supportive element is disposed between consecutive scaffolds, thereby defining the space separating therebetween. According to yet still further embodiments, each scaffold is placed between at least two supportive elements and is optionally coupled or attached thereto.

According to some embodiments, each scaffold of the plurality of scaffolds further comprises at least one structural element configured to separate the scaffold from its neighboring scaffolds. According to further embodiments, the at least one structural element is made of said scaffold material. According to certain embodiments, the at least one structural element forms an integral part of the scaffold.

According to some embodiments, the at least one scaffold unit comprising the plurality of scaffolds is disposed within the cell culture bioreactor, so that each of the scaffolds is positioned with its two surfaces being in parallel to a bottom surface of the bioreactor.

According to some embodiments, during the operation of the at least one pump, the liquid passes in parallel to the longitudinal axis within each space along at least one surface of each of the scaffolds, between neighboring scaffolds.

According to some embodiments, each one of the inlet manifold and the outlet manifold is formed as at least one liquid conduit comprising the plurality of openings located thereon. According to some embodiments, each one of the inlet and the outlet manifolds are formed as an integral part of a portion of the bioreactor comprising the plurality of openings located on a surface or a wall thereof.

According to some embodiments, the at least one scaffold unit is disposed within the internal chamber of the cell culture bioreactor, so that at least one of the openings of each manifold is positioned directly opposing a corresponding space between two neighboring or consecutive scaffolds of the scaffold unit, in order to enable direct liquid flow therethrough, from the inlet manifold into each space between neighboring scaffolds of the scaffold unit and into the outlet manifold. According to some embodiments, one or more openings of each manifold is located above an upper scaffold of the scaffold unit, configured to enable direct liquid flow therethrough above the upper scaffold. According to some embodiments, the at least one pump is configured to maintain a substantially constant liquid level within the cell culture bioreactor.

According to some embodiments, the flow rate of the liquid is controlled by the at least one pump in order to maintain a constant liquid level within the culture bioreactor.

According to some embodiments, the cultivation system comprises a plurality of pumps, wherein a first pump is configured to deliver liquid into the at least one cell culture bioreactor via the inlet manifold at an inlet flow rate, and a second pump is configured to collect used liquid from the cell culture bioreactor via the outlet manifold at an outlet flow rate. According to further embodiments, the cultivation system further comprises a third pump fluidly coupled to the bioreactor. According to still further embodiments, one or more of the first pump, the second pump, and optionally the third pump is configured to control liquid level within the bioreactor.

According to some embodiments, the inlet flow rate is above about 0.5 volume exchanges per day (VVD). According to some embodiments, the desired liquid level within the cell culture bioreactor is at least about 0.1 mm above an upper scaffold of the scaffold unit. According to further embodiments, the desired liquid level within the cell culture bioreactor is at least about 6 mm above the upper scaffold of the scaffold unit.

According to some embodiments, the cultivation system further comprises at least one sensor, wherein said at least one sensor is configured to measure one or more of: liquid level within the cell culture bioreactor, liquid presence within the cell culture bioreactor; and at least one liquid parameter selected from the group consisting of: temperature, pH, dissolved oxygen concentration, concentration of one or more nutrients, and concentration of one or more waste products. According to some embodiments, the cultivation system comprises at least one liquid level sensor configured to measure liquid level within the cell culture bioreactor and/or at least one liquid contact sensor configured to detect the presence of liquid within the bioreactor.

According to some embodiments, the cultivation system further comprises a control unit in operative communication with the at least one sensor, configured to receive measurements of one or more of the liquid level, presence, and the at least one liquid parameter, and adjust it based on the measurement thereof.

According to some embodiments, the liquid is a cell culture medium.

According to some embodiments, the system further comprises a medium reservoir for supplying homogenous cell culture medium into the at least one cell culture bioreactor, wherein the medium reservoir is in fluid communication with the at least one cell culture bioreactor, and wherein said medium reservoir contains a homogenous cell culture medium for the cultivation of cells and/or tissues. According to some embodiments, the medium reservoir is configured to supply to the at least one cell culture bioreactor a homogenous cell culture medium, configured to support the cultivation of cells and/or tissues. Any method/apparatus suitable for maintaining the homogeneity of the medium within the medium reservoir may be used with the teachings of the invention. According to certain exemplary embodiments, the medium reservoir is configured to mix/stir the medium. According to certain exemplary embodiments, the medium reservoir further comprises one or more sensors for measuring in the medium a medium parameter selected from the group consisting of, but not limited to, temperature, pH, dissolved oxygen concentration, concentration of one or more nutrients, concentration of one or more waste products, and any combination thereof. According to certain additional exemplary embodiments, the medium reservoir is configured to enable the adjustment of the at least one parameter based on the measurement thereof, thereby obtaining treated homogeneous medium which can be then circulated back into the bioreactor.

According to some embodiments, the system further comprises one or more of: a separation system configured to remove waste products from the medium entering thereto; a delivery system configured to circulate the medium within the system, and specifically to deliver the medium from the medium reservoir into the cell culture bioreactor via the inlet manifold and collecting used medium from said cell culture bioreactor via the outlet manifold, and optionally further configured to deliver the used medium to the separation system and the medium from said separation system to the culture medium reservoir; and combinations thereof.

According to some embodiments, the system further comprises a cell trap between the outlet of the cell culture bioreactor and an inlet to the medium reservoir.

According to some embodiments, the flow rate of the cell culture medium is controlled to support the cell cultivation on at least one surface of each of the scaffolds or therewithin. According to some embodiments, the flow rate is controlled according to a measure of at least one of medium level, dissolved oxygen, pH, nutrient concentration of one or more nutrients, concentration of one or more waste products, and any combination thereof.

According to some embodiments, the system comprises a plurality of cell culture bioreactors.

According to some embodiments, the cell culture bioreactor and/or the medium reservoir are selected from the group consisting of a stainless-steel vessel and a flexible bag. Each possibility represents a separate embodiment of the present invention.

According to some embodiments, the cells are non-human-animal cells. According to some embodiments, the system is for use in producing cultured food products.

According to some embodiments, the scaffolds comprise at least one edible material. According to some embodiments, the scaffolds are entirely made from at least one edible material.

According to another aspect, there is provided a method for producing at least one scaffold unit comprising a plurality of scaffolds comprising cultured cells and/or tissues thereon and/or therewithin, the method comprising the steps of: (a) providing at least one scaffold unit comprising a plurality of scaffolds, wherein each scaffold is separated from its neighboring scaffolds to form spaces therebetween enabling liquid flow, wherein each scaffold is porous.

The method further comprises step (b) seeding cells on at least part of a surface of each scaffold of the plurality of scaffolds and/or within each scaffold of the plurality of scaffolds.

The method optionally further comprises step (c) placing the scaffold unit comprising the plurality of scaffolds under conditions enabling adherence of the seeded cells thereto, until said cells are at least partially covering at least one surface of each of the scaffolds, or until the cells are covering at least a part of the pores and/or channels of each of the scaffolds.

The method further comprises step (d) placing the scaffold unit comprising the plurality of scaffolds having at least part of each scaffold seeded with said cells in a cell culture bioreactor comprising an inlet manifold and an outlet manifold, wherein each manifold comprises a plurality of openings configured to direct a flow of a liquid therethrough, wherein the manifolds are connected to at least one pump configured to control the flow rate of said liquid.

The method further comprises step (e) operating the at least one pump to deliver the liquid from the inlet manifold, along said spaces between neighboring scaffolds of at least one scaffold unit, and into the outlet manifold.

The method further comprises step (f) cultivating the cells on and/or within the plurality of scaffolds, thereby producing a plurality of scaffolds comprising cultured cells and/or tissues thereon and/or therewithin.

According to some embodiments, step (b) of seeding the cells comprises seeding cells on one or two of the surfaces of each scaffold.

According to some embodiments, step (b) of seeding the cells comprises administering the cells directly into each scaffold, or into the spaces separating neighboring scaffolds. According to further embodiments, each scaffold comprises pores and/or channels, and step (b) of seeding the cells comprises injecting the cells directly into at least part of the pores/channels of each scaffold.

According to some embodiments, step (d) comprises placing the scaffold unit in the cell culture bioreactor such that at least one opening of each manifold is positioned directly opposing a corresponding space separating consecutive or neighboring scaffolds of the scaffold unit, thereby enabling direct liquid flow therethrough.

According to some embodiments, step (e) comprises delivering the cell culture medium (i) from the plurality of openings of the inlet manifold into each space separating consecutive or neighboring scaffolds of the scaffold unit, (ii) from the spaces into the plurality of openings of the outlet manifold, and (iii) from the plurality of openings of the outlet manifold into the plurality of openings of the inlet manifold, thereby circulating the liquid within the bioreactor.

According to some embodiments, the at least one pump comprises a plurality of pumps, wherein during step (e) a first pump is delivering the liquid into the cell culture bioreactor via the inlet manifold at an inlet flow rate, a second pump is collecting used liquid from the cell culture bioreactor via the outlet manifold at an outlet flow rate, and optionally a third pump is fluidly coupled to the bioreactor, wherein one or more of the first pump, the second pump, and optionally the third pump control the liquid level within the cell culture bioreactor. According to some embodiments, the inlet flow rate is above about 0.5 volume exchanges per day (VVD). According to some embodiments, the desired liquid level within the cell culture bioreactor is at least about 0.1 mm above an upper scaffold of the scaffold unit.

According to some embodiments, step (f) comprises cultivating the cells on and/or within the plurality of scaffolds to reach at least one pre-set parameter selected from the group consisting of a desired tissue mass; nutrient uptake rate; oxygen uptake rate; waste production rate; and any combination thereof, thereby producing a plurality of scaffolds comprising cultured cells and/or tissues thereon and/or therewithin.

According to some embodiments, the nutrient is selected from the group consisting of glucose and glutamine.

According to some embodiments, the waste product is selected from the group consisting of ammonium and lactate.

According to some embodiments, each of the scaffolds comprises two surfaces facing opposite directions along the longitudinal axis of said scaffold. According to some embodiments, none of the surfaces defines an enclosed perimeter across any cross-section thereof. According to some embodiments, each of the scaffolds is planar. According to some embodiments, the liquid passes along the longitudinal axis of each scaffold of the plurality of scaffolds. According to some embodiments, the liquid passes on at least one surface of the scaffold, within the scaffold volume, or a combination thereof. According to some embodiments, the plurality of scaffolds is vertically stacked, one on top of the other, along and/or in parallel to a vertical axis, wherein each scaffold is spaced from the following scaffold by at least one supportive element.

According to some embodiments, the liquid is a cell culture medium.

According to some embodiments, the method is performed under aseptic conditions.

According to some embodiments, the cells are non-human-animal cells. According to some embodiments, the scaffold unit is edible.

According to some embodiments, the method further comprises a final step of removing at least one scaffold of the plurality of scaffolds comprising cultured cells and/or tissues from the cell culture bioreactor.

According to some embodiments, the method is used for producing a cultured food product. According to some embodiments, the cultured food product is cultured meat.

According to some embodiments, there is provided a scaffold unit comprising a plurality of scaffolds comprising cultured cells and/or tissues thereon and/or therewithin, produced by the method as disclosed herein above. According to further embodiments, there is provided a cultured food product comprising at least one scaffold unit as disclosed herein.

According to another aspect, there is provided a bioreactor configured to deliver a liquid to a plurality of scaffolds, the bioreactor comprising: (a) at least one bioreactor wall defining an internal chamber and is extending between a bottom surface and an upper surface and is positioned perpendicularly thereto, in parallel to a vertical axis; (b) at least one main tube, positioned along the vertical axis, and is extending perpendicularly from the upper surface; (c) a plurality of cultivation trays disposed within the internal chamber, wherein said plurality of cultivation trays extends radially from at least one supportive element directly or indirectly attached to the main tube, wherein each cultivation tray is configured to support at least one scaffold and is further configured to enable a liquid to pass along and/or through each scaffold; and (d) at least one mixing apparatus attached to the main tube, wherein the mixing apparatus is configured to circulate the liquid within the bioreactor, optionally in order to control the flow rate thereof within the bioreactor, and wherein the bioreactor is configured to deliver the liquid to the plurality of scaffolds.

According to some embodiments, the at least one supportive element is shaped as a hollow tube and encompasses the main tube, thus defining an inner supportive element space therebetween which is configured to enable the liquid to flow therethrough. According to some embodiments, the at least one supportive element comprises a plurality of openings spaced from each other along a circumference thereof. According to some embodiments, a portion of the plurality of openings is located above each respective cultivation tray. According to some embodiments, each cultivation tray comprises at least one scaffold disposed therein or thereon.

According to some embodiments, the plurality of cultivation trays extends radially from a corresponding plurality of supportive elements fluidly coupled to one another, and wherein each supportive element is attached to at least one cultivation tray. According to some embodiments, the plurality of supportive elements is fluidly coupled to one another, wherein each supportive element is shaped as a hollow tube, thus defining an inner supportive element space therebetween, wherein the liquid can flow through. According to some embodiments, each supportive element comprises a plurality of openings spaced from each other along a circumference thereof. According to some embodiments, a portion of the plurality of openings is located above each respective cultivation tray.

According to some embodiments, each cultivation tray comprises a bottom tray and an upper tray, supporting at least one scaffold disposed therebetween. According to some embodiments, each one of the bottom tray and the upper tray comprises a mesh and/or a porous structure, in order to enable the fluid to pass therethrough and/or therealong and to contact the at least one scaffold disposed therebetween.

According to some embodiments, each cultivation tray is planar. According to some embodiments, each cultivation tray is disc shaped or circular shaped.

According to some embodiments, the mixing apparatus is located in the vicinity of the bottom surface. According to some embodiments, the mixing apparatus is configured to circulate the liquid within the bioreactor, in order to control the flow rate of the liquid passing through and/or along the plurality of scaffolds.

According to some embodiments, the liquid is selected from a cell-suspension medium comprising a plurality of cells to be seeded on and/or within the scaffolds, a cell culture medium supporting the cultivation of cells and/or tissues, and an aqueous solution. According to certain embodiments, the aqueous solution is a buffer. The buffer may be used to wash the bioreactor internal space and/or scaffolds.

According to some embodiments, the scaffolds are made of porous materials. According to some embodiments, the scaffolds are edible.

According to some embodiments, there is provided a method for producing a plurality of scaffolds comprising cultured cells and/or tissues thereon, comprising the steps of: (a) providing the bioreactor as disclosed herein above; (b) seeding cells on and/or within at least a part of each scaffold of the plurality of scaffolds, utilizing a cell-suspension medium comprising a plurality of cells to be seeded on and/or within the scaffolds, thereby obtaining cell-seeded scaffolds, wherein the seeding is performed within the bioreactor of step (a) or outside thereof; (c) circulating a cell culture medium within the bioreactor, wherein the cell-seeded scaffolds is placed within the bioreactor prior to step (c), wherein the cell culture medium is configured to support the cultivation of cells seeded on and/or within the scaffolds and cells/tissues developed therefrom; and (d) cultivating the cells on and/or within the plurality of scaffolds, thereby producing a plurality of scaffolds comprising cultured cells and/or tissues.

According to some embodiments, step (d) comprises cultivating the cells on and/or within the plurality of scaffolds to reach at least one pre-set parameter selected from the group consisting of a desired tissue mass; nutrient uptake rate; oxygen uptake rate; waste production rate; and any combination thereof, thereby producing a plurality of scaffolds comprising cultured cells and/or tissues.

According to some embodiments, step (b) comprises flowing the cell-suspension medium into the bioreactor and rotating the mixing apparatus in order to circulate the cell suspension medium therein, wherein optionally the circulation of the cell-suspension medium within the bioreactor causes the seeding of the cells on the plurality of scaffolds, or alternatively wherein the seeding is performed outside of the bioreactor.

According to some embodiments, the cell suspension medium is circulated within the bioreactor by flowing from the plurality of openings of each supportive element, through and/or along the plurality of scaffolds supported by each respective cultivation tray, onwards through the rotating mixing apparatus, towards the inner supportive element space defined by the plurality of supportive elements, and so on.

According to some embodiments, step (b) comprises flowing the cell-suspension medium into the bioreactor, wherein the flowing of the cell-suspension medium into the bioreactor causes the seeding of the cells on the plurality of scaffolds. According to some embodiments, after the cells were seeded on the plurality of scaffolds in step (b) and prior to step (c), if the cell culture medium is different from the cell-suspension medium, the method further comprises exchanging the cell-suspension medium with the cell culture medium.

According to some alternative embodiments, step (b) comprises seeding the cells on and/or within the plurality of scaffolds outside of the bioreactor, wherein step (c) initially comprises placing the cell-seeded scaffolds within the bioreactor, preferably prior to circulating a cell culture medium within the bioreactor.

According to some embodiments, step (c) comprises rotating the mixing apparatus in order to circulate the cell culture medium within the bioreactor.

According to some embodiments, the method further comprises a final step of removing at least one scaffold comprising cultured cells and/or tissues from the bioreactor.

According to some embodiments, the nutrient is selected from the group consisting of glucose and glutamine. According to some embodiments, the waste product is selected from the group consisting of ammonium and lactate. According to some embodiments, the cells are non-human-animal cells. According to some embodiments, said method is for producing a cultured food product. According to some embodiments, the cultured food product is cultured meat.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawings will be provided by the Office upon request and payment of the necessary fee.

Some embodiments of the invention are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the invention. For the sake of clarity, some objects depicted in the figures are not to scale.

In the Figures:

FIGS. 1A-1E are functional block diagrams depicting a system 100, according to different embodiments of the present invention.

FIG. 2A is a view in perspective of a scaffold unit 120, according to some embodiments.

FIG. 2B is a view in perspective of some components of a bioreactor 110, according to some embodiments.

FIG. 2C is a view in perspective of a manifold, according to some embodiments.

FIG. 3A is a cross sectional view of a scheme of a bioreactor 210, according to some embodiments.

FIG. 3B is a cross sectional view in perspective of bioreactor 210, according to some embodiments.

FIGS. 3C-3E are views in perspective of some components of bioreactor 210, according to some embodiments.

FIG. 4 is a view in perspective of the bioreactor 110 symmetry plan.

FIGS. 5A and 5B demonstrate the effect of medium level above the top scaffold at a flow rate of 1.5 vessel volume exchanges per day (VVD). The color bar on the left is oxygen concentration in mmol/mi while the color bar on the right is velocity in m/sec. FIG. 5A: the high-level medium configuration shows circulation at the inlet, top scaffold and outlet, and low oxygen concentration unevenly distributed on all scaffolds. FIG. 5B: the low-level medium configuration shows circulation at the inlet only and oxygen concentration evenly distributed on all scaffolds with no zero concentration zones.

FIG. 6 demonstrates the continuous pH, dissolved oxygen (DO) and temperature measurements at the outlet of the culture bioreactor of Example 3.

FIG. 7 presents the cell cultivation on a scaffold at the end of the cultivation period, according to the protocol described at Example 3.

### DETAILED DESCRIPTION OF THE INVENTION

The present invention provides systems and methods for advanced cell and/or tissue cultivation, optionally for the production of cultured food products.

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure. In the figures, like reference numerals refer to like parts throughout.

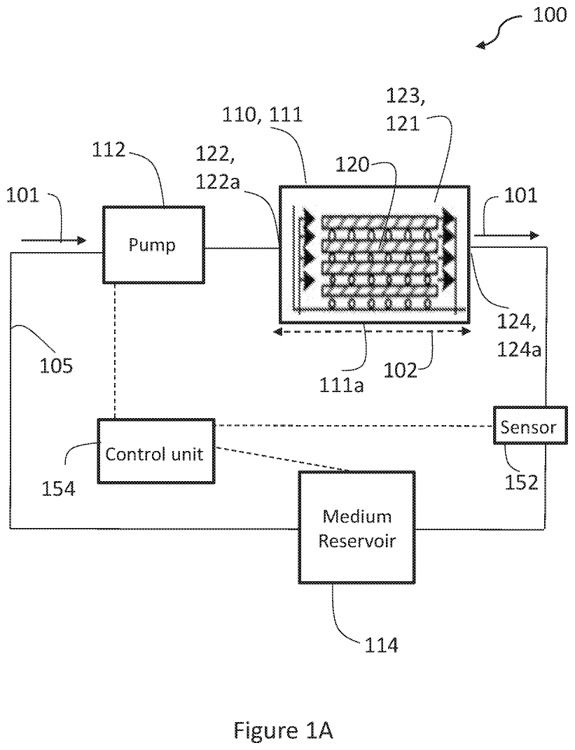

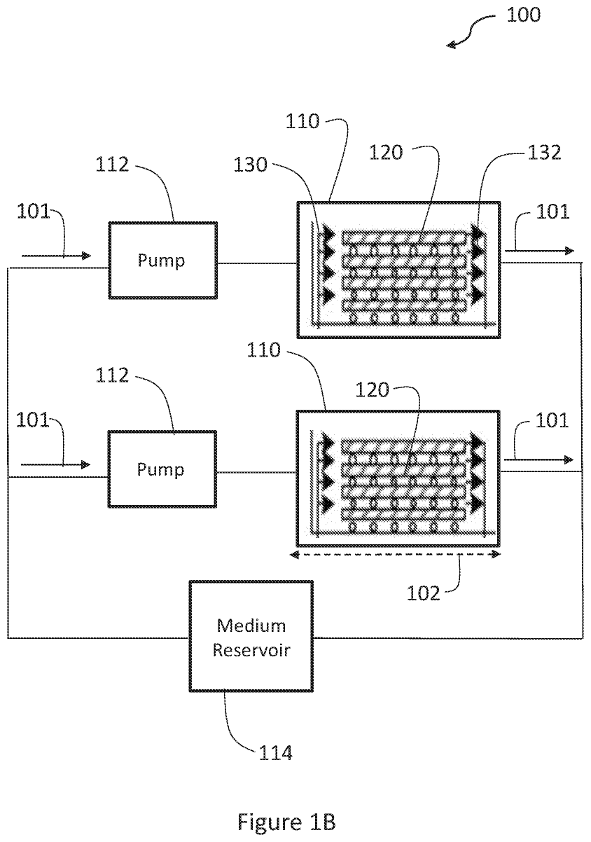

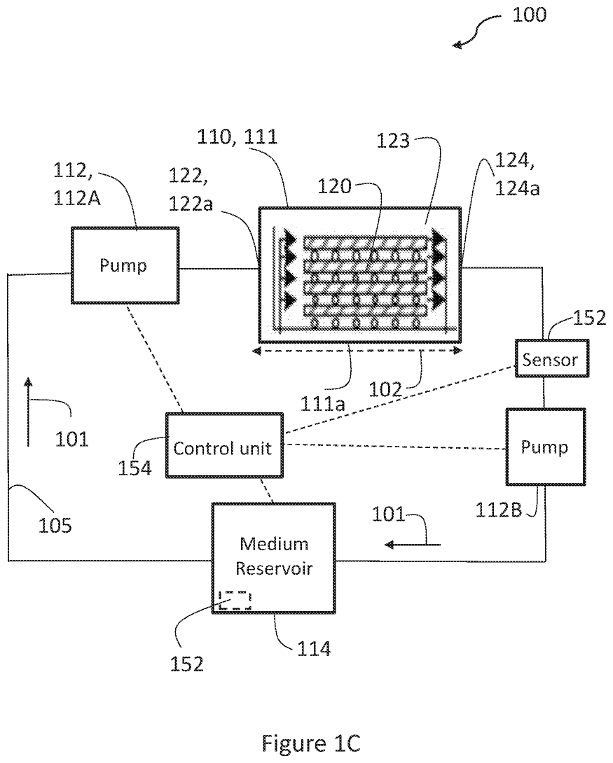

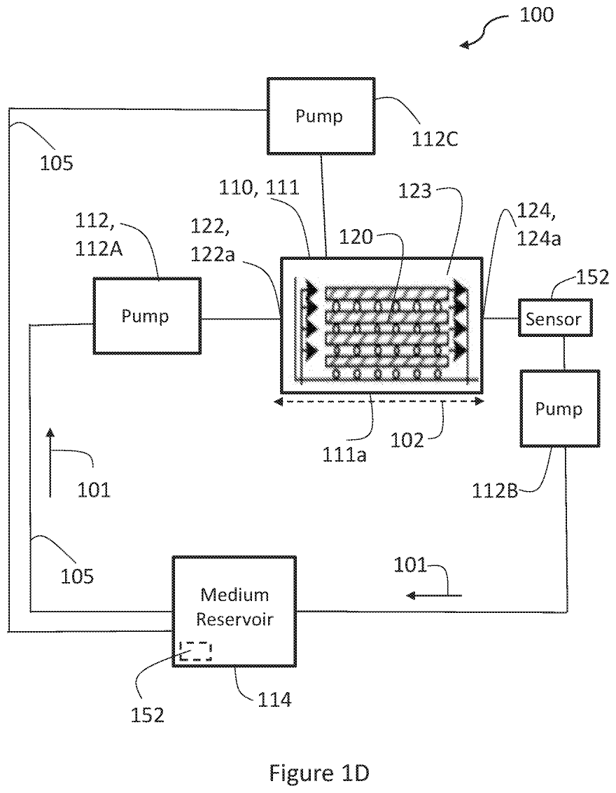

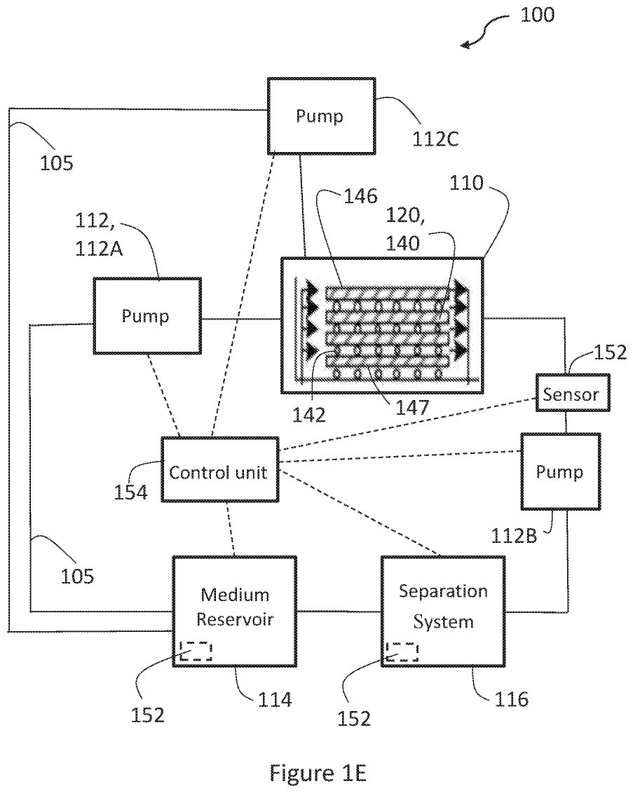

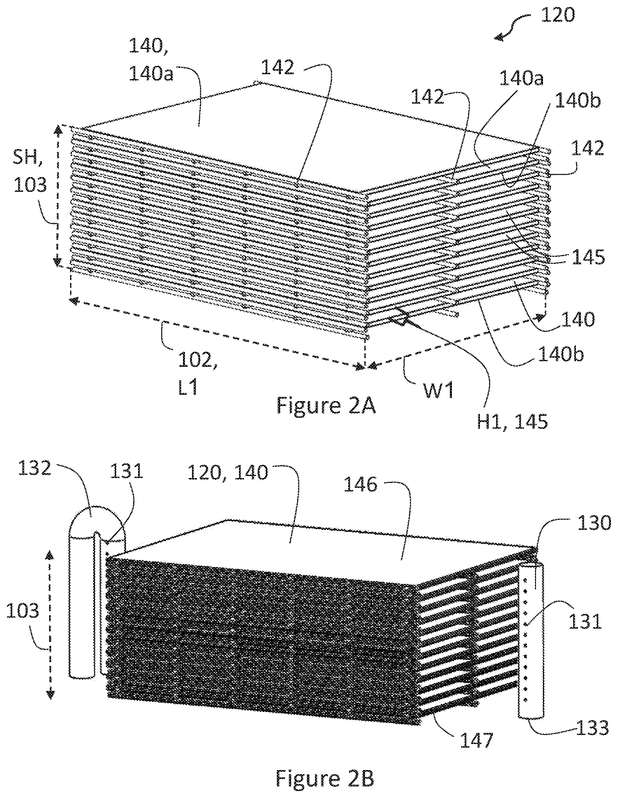

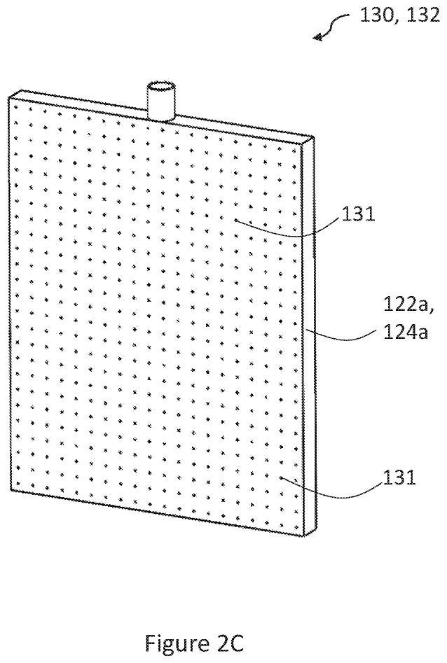

Reference is now made to FIGS. 1A-2C. FIGS. 1A-1E are functional block diagrams depicting a system 100, according to different embodiments of the present invention. FIG. 2A is a view in perspective of a scaffold unit 120, according to some embodiments. FIG. 2B is a view in perspective of components of a bioreactor 110, according to some embodiments. FIG. 2C is a view in perspective of a manifold, according to some embodiments.

According to a first aspect, there is provided a cultivation system 100 configured to deliver a liquid to a population of cultured cells and/or tissues. According to further embodiments, the system 100 is configured to deliver said liquid to said population of cultured cells and/or tissues, in order to support the cultivation of cells and enable the large-scale production of cultured food products at a cost-effective manner. As used herein, the terms “cell cultivation” or “cultivation of cells” refers to any and all stages of cell cultivation, including cell growth (proliferation), differentiation and maturation.

According to some embodiments, the cultivation system 100 is configured to support and/or enable cell and/or tissue cultivation on and/or within at least one three-dimensional (3D) multi-layer scaffold unit 120, wherein said scaffold unit 120 is appropriate for supporting seeding, growth and expansion and/or differentiation of the cells and/or tissues thereon and/or therewithin. According to some embodiments, the cultivation system 100 is configured to support and/or enable cell and/or tissue cultivation on and/or within at least one three-dimensional (3D) multi-layer scaffold unit 120, wherein said scaffold unit 120 is appropriate for supporting seeding, growth and expansion and/or differentiation of the cells and/or tissues thereon and/or therewithin and further production of cell products by the cultivated cells/tissue. According to certain embodiments, the cell products are proteins. According to some embodiments, the protein is collagen.

According to certain embodiments, the scaffold unit 120 is appropriate for supporting production of cell products by the cultivated cells/tissue and further the secretion of the compounds produced by the cells and/or tissue into the liquid.

According to some embodiments, the cultivation system 100 is configured to support and/or enable cell and/or tissue cultivation on a plurality of three-dimensional (3D) scaffold units 120.

As used herein, the term “plurality” refers to “at least two”.

According to some embodiments, the cultivation system 100 comprises at least one cell culture bioreactor 110, as illustrated at FIG. 1A. According to further embodiments, the cultivation system 100 comprises at least two cell culture bioreactors 110, as illustrated at FIG. 1B. According to some embodiments, the cultivation system 100 comprises a plurality of culture bioreactors 110.

According to some embodiments, the cultivation system 100 comprises at least one cell culture bioreactor 110 in fluid communication with at least one medium reservoir 114 in fluid communication with at least one pump 112, wherein the at least one pump 112 is configured to circulate the liquid within the cultivation system 100. In some embodiments, the bioreactor 110, the at least one pump 112, and the medium reservoir 114 are fluidly coupled to one another via fluid conduits 105 (e.g., tubes or pipes) configured to enable fluid flow therethrough. In some embodiments, the medium reservoir 114 comprises the liquid disposed therein, wherein the liquid is a medium, typically cell cultivation medium. In some embodiments, the bioreactor 110 and the at least one pump 112 are fluidly coupled to each other via at least one fluid conduit 105, wherein the fluid conduit 105 is configured to enable fluid flow therethrough. In some embodiments, the medium reservoir 114 and the bioreactor 110 and/or the at least one pump 112 are fluidly coupled to each other via at least one fluid conduit 105.

As used herein, the term “fluid communication” refers to a path which allows fluid to flow between two components, wherein said two components can be directly or indirectly joined to each other. Similarly, as used herein, the terms “fluidly coupled” or “fluidly connected” are interchangeable, and refers to a connection between two components that allows fluid to flow from one component to the other, wherein said connection may be direct or indirect via an intermediate component enabling fluid flow therethrough. A variety of complementary structures are known in the art for fluid coupling. Among these include pipes, tubes, chambers, containers, reservoirs, electric appliances (e.g., a pump), adaptors, and the like.

According to some embodiments, the at least one cell culture bioreactor 110 has a three-dimensional (3D) structure. According to some embodiments, the bioreactor 110 has a shape or a structure adapted to accommodate within the at least one scaffold unit 120. The cell culture bioreactor 110 of the present invention is also referred to herein as cultivator or cultivating chamber. According to some embodiments, the cell culture bioreactor 110 is configured for producing or manufacturing cultivated meat products.

According to some embodiments, the bioreactor 110 comprises an inlet port 122, an outlet port 124, and at least one bioreactor wall 111 defining an internal chamber 123. According to further embodiments, the bioreactor 110 comprises a plurality of bioreactor walls 111 defining the internal chamber 123. According to further embodiments, the internal chamber 123 defines an internal chamber space 121 in which the at least one scaffold unit 120 is placed within.

According to some embodiments, the bioreactor 110 comprises a first surface 122a comprising the inlet port 122, and a second surface 124a positioned substantially parallel thereto, comprising the outlet port 124. According to some embodiments, the first surface 122a and/or the second surface 124a of the bioreactor 110 are bioreactor walls. According to some embodiments, the at least one bioreactor wall 111 is positioned perpendicularly to the first surface 122a and to the second surface 124a. According to some embodiments, the at least one bioreactor wall 111 is extending from the first surface 122a towards the second surface 124a. According to some embodiments, the bioreactor 110 comprises a plurality of walls 111, wherein each one is extending from the first surface 122a to the second surface 124a. The bioreactor 110 or the at least one bioreactor wall 111 thereof can be shaped as a cylinder, a box, a sphere, or any other suitable shape in the art. Each possibility represents a different embodiment.

According to some embodiments, the bioreactor 110 comprise a bottom surface 111a, extending from the first surface 122a to the second surface 124a, optionally in parallel to a longitudinal axis 102. According to some embodiments, the bioreactor 110 comprise a top surface extending from the first surface 122a to the second surface 124a.

According to some embodiments, the inlet port 122 is in fluid communication with the outlet port 124 via the internal chamber space 121 defined by the internal chamber 123. According to some embodiments, the at least one scaffold unit 120 is disposed within the bioreactor 110. According to some embodiments, the at least one scaffold unit 120 is disposed within the internal chamber 123, and specifically within the internal chamber space 121. According to some embodiments, a plurality of scaffold units 120 are disposed within the bioreactor 110.

As used herein, the term “substantially” refers to the complete or nearly complete extent or degree of a characteristic property. For example, a substantially parallel surface may be a parallel surface, or a surface that is inclined in an angle that is bellow about 5°.

According to some embodiments, each scaffold unit 120 has dimensions comprising a length L1, a width W1, and a scaffold unit height SH (see FIG. 2A).

According to some embodiments, the length L1 is identical to the width W1, or is different therefrom.

According to some embodiments, the length L1 is above about 1 cm, above about 10 cm, above about 20 cm, above about 30 cm, above about 40 cm, above about 50 cm, above about 60 cm, above about 70 cm, above about 80 cm above bout 90 cm, 100 cm, above about 5 m, above about 10 m, or more. Each possibility represents a different embodiment. According to some embodiments, the length L1 ranges from about 1 cm to about 100 cm. According to further embodiments, the length L1 ranges from about 1 cm to about 25 cm, or optionally from about 5 cm to about 15 cm.

According to some embodiments, the width W1 is above about 1 cm, above about 10 cm, above about 20 cm, above about 30 cm, above about 40 cm, above about 50 cm, above about 60 cm, above about 70 cm, above about 80 cm above bout 90 cm, or 100 cm, above 5 m, above 10 m, or more. Each possibility represents a different embodiment. According to some embodiments, the width W1 ranges from about 1 cm to about 100 cm. According to further embodiments, the width W1 ranges from about 1 cm to about 25 cm, or optionally from about 1 cm to about 10 cm.

According to some embodiments, the cultivation system 100 or parts thereof (e.g., medium reservoir 114) can be operated in an operation mode selected from at least one of a batch, fed batch, perfusion, and any combination thereof. Each possibility represents a different embodiment.

According to some embodiments, the cell culture bioreactor 110 comprise a plug flow reactor, a wave bioreactor, any other suitable bioreactor in the art, or a combination thereof. Each possibility represents a different embodiment. According to some embodiments, the cell culture bioreactor 110 is an unmixed tank.

As used herein, the terms “perfusion mode” or “open loop perfusion configuration” are interchangeable, and refers to a bioreactor system which is able to continuously feed cells disposed and cultured therein with fresh or treated media while remove spent or used media. By continuously removing spent medium from the system and replacing it with new or treated medium, nutrient levels within the bioreactor are maintained for optimal cultivation conditions, while cell waste products are removed in order to avoid toxicity.

According to some embodiments, the medium reservoir 114 is configured to be operated in a perfusion mode, wherein spent medium exits the bioreactor 110, replaced with new or treated medium within the medium reservoir 114, and is then circulated into the bioreactor 110. According to further embodiments, spent medium existing the bioreactor 110 is continuously replaced with new or treated medium within the medium reservoir 114, while simultaneously medium is continuously circulated into the bioreactor 110.

According to some embodiments, the bioreactor 110 and/or medium reservoir 114 are selected from a stainless-steel vessel and a flexible bag.

According to some embodiments, the scaffold unit 120 comprises a plurality of scaffolds 140, wherein each one of the plurality of scaffolds 140 constitutes as a single layer within the scaffold unit 120. According to some embodiments, each one of the plurality of scaffolds 140 is characterized by having dimensions comprising the length L1, the width W1, and a scaffold thickness positioned in parallel to the vertical axis 103. Alternatively, according to some embodiments, the length and/or the width of each one of the plurality of scaffolds 140 can be characterized by having dimensions slightly shorter than compared to the length L1 and/or the width W1. According to some embodiments, each scaffold unit 120 extends from a bottom scaffold 147 towards an upper scaffold 146 (see FIG. 2B), in parallel to the vertical axis 103, wherein each of the bottom scaffold 147 and the upper scaffold 146 is identical to each scaffold 140. According to further embodiments, the scaffold unit height SH is defined between the bottom scaffold 147 and the upper scaffold 146, in parallel to the vertical axis 103.

As used herein, the term “slightly shorter” refers to a dimension which is smaller by at least 0.1%, optionally at least 1%, or alternately at least 5%, or more, of a specified value.

According to some embodiments, the scaffold comprises at least one edible material. According to certain exemplary embodiments, the entire scaffold is edible.

According to some embodiments, at least some of the scaffolds 140 comprises a population of cells adhered thereto or cultured thereon and/or therewithin. According to some embodiments, each scaffold 140 comprises a population of cells adhered thereto. According to some embodiments, each scaffold 140 comprises a population of cells cultured thereon and/or therewithin. According to some embodiments, each scaffold 140 is appropriate for supporting seeding, and cultivation of cells and/or tissues thereon and/or therewithin.

According to some embodiments, each scaffold 140 is separated from its neighboring or adjacent scaffold 140 to form a space 145 along which the liquid (e.g., a cultivation medium) may pass/flow, optionally wherein at least part of a surface of each scaffold is covered with cells seeded thereon.

According to some embodiments, the plurality of scaffolds 140 are aligned in the same direction, in parallel to a longitudinal axis 102. According to further embodiments, the plurality of scaffolds 140 are vertically stacked, one on top of the other, along and/or in parallel to a vertical axis 103, wherein each scaffold 140 is separated or spaced from a following scaffold 140 to form a space 145 therebetween, wherein each space 145 is defined by a space height H1. Said space height H1 represents the distance or height in parallel to the vertical axis 103 between consecutive or following scaffolds 140. According to some embodiments, consecutive or following scaffolds 140 are evenly spaced from each other, thereby forming identical spaces 145 therebetween, wherein said identical spaces 145 all have the same space height H1.

Advantageously, each space 145 between consecutive or following scaffolds 140 can enable the liquid (e.g., a cultivation medium) to flow/pass between neighboring scaffolds 140 and to form contact therewith and/or therealong, in order to support the growth and/or expansion and/or differentiation of cells and/or tissues, cultured on and/or within the plurality of scaffolds 140. According to some embodiments, the spaces 145 can enable the uniform distribution of the liquid passing between neighboring scaffolds 140, optionally along the entire surface thereof.

According to some embodiments, the space height H1 between consecutive scaffolds 140, in parallel to the vertical axis 103 defining the height of each space 145, is below about 100 mm, preferably below about 50 mm, or more preferably below about 30 mm. According to further embodiments, the space height H1 formed between neighboring scaffolds ranges from 0.1 mm to 10 mm. According to some embodiments, the height of each space 145 formed between two neighboring scaffolds in parallel to the vertical axis 103 (i.e., space height H1) is below about 30 mm, optionally below about 15 mm, or alternatively below about 8 mm. According to further embodiments, the height of each space 145 formed between neighboring scaffolds in parallel to the vertical axis 103 (i.e., space height H1) ranges from 0.1 mm to 15 mm, alternatively ranges from 3 mm to 10 mm, or optionally ranges from 5 mm to 8 mm. Each possibility represents a different embodiment. According to exemplary embodiments, the height of each space 145 formed between neighboring scaffolds in parallel to the vertical axis 103 (i.e., space height H1) is about 6 mm.

According to some embodiments, the scaffold unit 120 further comprises a plurality of supportive elements 142 separating each scaffold 140 from its neighboring scaffolds. According to further embodiments, the plurality of supportive elements 142 is configured to provide support to the plurality of scaffolds 140 and to maintain the space height H1 defining each space 145 spacing therebetween, in order to enable the liquid to pass/flow therethrough to support the growth and/or expansion and/or differentiation of cells and/or tissues, cultured thereon.

According to some embodiments, the plurality of scaffolds 140 are vertically stacked, one on top of the other, along and/or in parallel to the vertical axis 103, wherein each scaffold 140 is spaced from the following scaffold 140 by at least one supportive element 142. According to some embodiments, at least one supportive element 142 is disposed between consecutive scaffolds 140, thereby forming each space 145 defined by the space height H1 spacing therebetween. According to some embodiments, each scaffold 140 and at least one supportive element 142 are disposed alternately one over the other, so that the space 145 defined by the space height H1 is formed between each two consecutive scaffolds 140. According to some embodiments, the scaffold unit 120 comprises a plurality of spaces 145, wherein each space 145 is formed between each two consecutive scaffolds 140 and between two or more of supportive elements 142.

According to some embodiments, each scaffold 140 is at least partially placed onto at least one supportive element 142, wherein the supportive element 142 is positioned in parallel or vertically to the longitudinal axis 102. According to some embodiments, each scaffold 140 is placed between at least two, at least three, at least four, or more, supportive elements 142. According to some embodiments, a plurality of supportive elements 142 are disposed vertically to the longitudinal axis 102, between subsequent or consecutive scaffolds 140, thereby maintaining the space height H1 spacing therebetween, in order to enable the liquid to pass/flow horizontally therebetween to support the growth and/or expansion and/or differentiation of cells and/or tissues, cultured thereon and/or therewithin. According to some embodiments, the plurality of supportive elements 142 are disposed in parallel to the longitudinal axis 102, between subsequent scaffolds 140, thereby maintaining the space height H1. According to some embodiments, each one of the plurality of supportive elements 142 can be disposed between subsequent scaffolds 140 in various types of orientations and/or various angles relative to the longitudinal axis 102, in order to maintain the space height H1.

According to some embodiments, each scaffold 140 is attached to at least two supportive elements 142 located at opposite edges thereof, wherein at least one additional supportive element 142 is disposed in parallel to the longitudinal axis 102 between subsequent scaffolds 140, thereby maintaining the space height H1. According to some embodiments, each scaffold 140 is attached to at least two supportive elements 142 located at opposite edges thereof, wherein at least one additional supportive element 142 is disposed vertically to the longitudinal axis 102 between subsequent scaffolds 140, thereby maintaining the space height H1.

According to some embodiments, each supportive element 142 is elongated, that is the long dimension thereof (e.g., length) is greater than the short dimension (e.g., width or diameter) thereof. For example, the length of said elongated supportive element 142 may be at least three times, at least five times, at least ten times, or more, greater than of the width or diameter thereof. According to some embodiments, each supportive element 142 is a fiber. According to some embodiments, each supportive element 142 is shaped as an elongated tube or a rod, as illustrated at FIG. 2A. According to some embodiments, each supportive element 142 is made of the same materials and/or has the same properties and/or diameter (i.e., scaffold thickness) as each scaffold 140, as disclosed herein. According to some embodiments, a plurality of elongated rod-shaped supportive elements 142 are disposed between subsequent scaffolds 140.

According to some embodiments, at least one elongated supportive element 142 is disposed vertically or in parallel to the longitudinal axis 102, between subsequent or consecutive scaffolds 140, thereby providing support thereto and maintaining the space height H1 spacing between each two consecutive scaffolds 140. According to further embodiments, a plurality of elongated supportive elements 142 are disposed vertically or in parallel to the longitudinal axis 102, between subsequent or consecutive scaffolds 140, thereby providing support thereto and maintaining the space height H1 spacing between each two consecutive scaffolds 140. According to some embodiments, each supportive element 142 is coupled or attached to the consecutive scaffolds 140 it separates.

According to some embodiments, each scaffold 140 is attached to at least two elongated supportive elements 142 located at opposite edges thereof and thereby providing support thereto, wherein a plurality of additional supportive elements 142 are disposed in parallel and/or vertically to the longitudinal axis 102 between consecutive scaffolds 140, thereby spacing therebetween (see FIGS. 2A-B).

According to some embodiments, each scaffold 140 further comprises at least one structural element, configured to separate between subsequent scaffolds 140 (not shown). According to further embodiments, the at least one structural element is made of the same material(s) as the scaffold 140 is made from. The at least one structural element can maintain the space height H1 spacing between subsequent scaffolds 140, instead of the at least one supportive element 142, or in addition thereto. The at least one structural element can be integrally formed with each scaffold 140. According to certain embodiments, the scaffold and the structural elements are integrally formed by 3D printing.

Alternatively or additionally, in some embodiments, the scaffold unit 120 is disposed within (or is enveloped by) a support structure (not shown) configured to support and maintain the space height H1 between consecutive scaffolds 140, such as a frame or a surrounding housing (not shown). Said support structure may contain the plurality of supportive elements 142 as disclosed above, optionally in addition to the other supporting element(s). Alternatively, the support structure and/or the scaffold unit 120 does not contain the plurality of supportive elements 142.

According to some embodiments, the cell culture bioreactor 110 further comprises at least one inlet manifold 130 and at least one outlet manifold 132 disposed therein. According to further embodiments, each manifold comprises a plurality of openings 131 positioned therealong, wherein the openings 131 are spaced apart from each other along a surface or a wall of each manifold.

According to some embodiments, the plurality of openings 131 are extending through each manifold, i.e., the plurality of openings 131 are located on, or embedded within, a surface or a wall of each manifold and are configured to enable liquid flow between an internal space of each manifold and the scaffold unit 120 (disposed within the cell culture bioreactor 110). In some embodiments, the openings 131 are in the shape of a circle, ellipse, square, rectangle, or any other polygon.

The terms “located on” and “embedded within” as used herein collectively refer to the configuration of the openings 131 disclosed herein with respect to the surface of each manifold. The opening may be formed as an integral part of the surface, or generated on the surface (e.g., by puncturing or melting, for example, using laser) such that the resulting opening at the surface is preferably forming a continuous smooth and seamless surface with one or more openings.

According to some embodiments, a plurality of openings 131 extends through (i.e., located on, or embedded within) the inlet manifold 130, thereby enabling fluid communication and/or fluid flow between an internal space of the manifold and the internal chamber 123 of the bioreactor 110. According to some embodiments, a plurality of openings 131 extends through the outlet manifold 132, thereby enabling fluid communication between an internal space of the manifold and the internal chamber 123 of the bioreactor 110.

According to some embodiments, the inlet manifold 130 is fluidly connected to the inlet port 122, and the outlet manifold 132 is fluidly connected to the outlet port 124.

According to some embodiments, the inlet manifold 130 and the outlet manifold 132 are formed as integral parts of portions of the bioreactor 110, or as separate components therefrom.

According to some embodiments, the inlet manifold 130 is integrally formed with the inlet port 122, and the outlet manifold 132 is integrally formed with the outlet port 124. According to other embodiments, the inlet manifold 130 comprises an adaptor 133 connected to at least one manifold opening (see FIG. 2B), wherein said adaptor 133 is fluidly connected to the inlet port 122 and/or the at least one pump 112. According to some embodiments, the outlet manifold 132 comprises an adaptor 133 connected to at least one manifold opening, wherein said adaptor 133 is fluidly connected to the outlet port 124 and/or the at least one pump 112.

The term “integrally formed” as used herein refer to a body that is manufactured integrally, i.e., as a single piece, without requiring the assembly of multiple pieces. Multiple parts may be integrally formed with each other if they are formed as a single piece.

According to some embodiments, the inlet manifold 130 and the outlet manifold 132 are liquid conduits (e.g., tubes or pipes) configured to enable fluid flow therethrough (via an internal space of each manifold), each comprising a plurality of openings 131 extending therethrough, as illustrated for example, at FIG. 2B.

According to alternative embodiments, the inlet manifold 130 is integrally formed with a bioreactor wall, and the outlet manifold 132 is integrally formed with an opposing bioreactor wall. According to some embodiments, the inlet manifold 130 is integrally formed with the first surface 122a of the bioreactor 110, and the outlet manifold 132 is integrally formed with the second surface 124a thereof, as illustrated for example, at FIG. 2C. According to some embodiments, the inlet manifold 130 is integrated within the first surface 122a of the bioreactor 110, and the outlet manifold 132 is integrated within the second surface 124a thereof. According to some embodiments, the inlet manifold 130 is disposed within the first surface 122a of the bioreactor 110, and the outlet manifold 132 is disposed within the second surface 124a thereof.

According to some embodiments, the plurality of openings 131 of the inlet manifold 130 are located on, or embedded within, the first surface 122a of the bioreactor 110, and the plurality of openings 131 of the outlet manifold 132 are located on, or embedded within, the second surface 124a thereof, as illustrated for example, at FIG. 2C. According to further embodiments, liquid may flow from the inlet port 122, through the first surface 122a (comprising the inlet manifold 130 disposed therein), into the internal chamber space 121 via the plurality of openings 131, and then into the second surface 124a (comprising the outlet manifold 132 disposed therein) via the plurality of openings 131, and finally into the outlet port 124.

According to other embodiments, each of the inlet manifold 130 and the outlet manifold 132 comprise a main liquid conduit fluidly coupled to at least two conduit arms, wherein each arm comprises the plurality of openings 131 extending therethrough (not shown).

In some embodiments, the plurality of openings 131 of each manifold are configured to enable liquid flow therethrough and into the scaffold unit 120 disposed within the cell culture bioreactor 110. In some embodiments, the plurality of openings 131 extending through the inlet manifold 130 enable liquid flow therefrom and into the plurality of spaces 145, wherein each space 145 spaces consecutive scaffolds 140. In further embodiments, the liquid flows through consecutive scaffolds 140 within each space 145 and into the plurality of openings 131 extending through the outlet manifold 132.

According to some embodiments, the manifolds are fluidly connected to at least one pump 112 (see FIG. 1A), wherein the at least one pump 112 is configured to control the flow rate of the liquid within the system 100, and optionally to circulate the liquid within the system 100, during the operation thereof. According to some embodiments, the manifolds are fluidly coupled or connected to at least one pump 112, configured to control the flow rate of the liquid from the inlet manifold 130, through the scaffold unit 120, and into the outlet manifold 132.

According to alternative embodiments, the manifolds are fluidly connected to a plurality of pumps 112 (see FIGS. 1C-1E).

According to some embodiments, the at least one pump 112 is configured to direct the flow of the liquid from the inlet manifold 130, through the plurality of spaces 145 (or heights H1) separating consecutive scaffolds 140 of the scaffold unit 120, and into the outlet manifold 132, in parallel to a flow direction 101 (parallel to the longitudinal axis 102). According to further embodiments, the at least one pump 112 is configured to direct the flow of the liquid from the inlet port 122 into the inlet manifold 130, through the scaffold unit 120, into the outlet manifold 132 and onwards into the outlet port 124.

According to some embodiments, the at least one bioreactor wall 111 is extending from the first surface 122a to the second surface 124a, in parallel to the longitudinal axis 102, as illustrated at FIGS. 1A-1E. According to further embodiments, the scaffold unit 120 is disposed within the bioreactor 110 so that the length L1 thereof is positioned in parallel to the longitudinal axis 102. According to still further embodiments, the plurality of scaffolds 140 are disposed within the bioreactor 110 so that the length L1 of each scaffold 140 is positioned in parallel to the longitudinal axis 102. According to some embodiments, the at least one pump 112 is configured to direct the flow of the liquid from inlet manifold 130, through the plurality of spaces 145 (or heights H1) separating consecutive scaffolds 140 of the scaffold unit 120, and into the outlet manifold 132, in parallel to a flow direction 101 and to the longitudinal axis 102. According to some embodiments, the liquid passes or flows along the longitudinal axis 102 through the scaffold unit 120 such that it flows between neighboring scaffolds 140 through the plurality of spaces 145 separating therebetween.

According to some other embodiments, the at least one bioreactor wall 111 is extending from the first surface 122a to the second surface 124a, in parallel to the vertical axis 103 (not shown). According to further embodiments, the scaffold unit 120 is disposed within the bioreactor 110 so that the length L1 thereof is in parallel to the vertical axis 103. According to still further embodiments, the plurality of scaffolds 140 are disposed within the bioreactor 110 so that the length L1 of each scaffold 140 is positioned in parallel to the vertical axis 103. According to some embodiments, the at least one pump 112 is configured to direct the flow of the liquid from inlet manifold 130, through the scaffold unit 120, and into the outlet manifold 132, in parallel to the vertical axis 103. According to some embodiments, the liquid passes along the vertical axis 103 between neighboring scaffolds 140, in the direction of the gravity force.

According to some embodiments, the liquid passes in parallel to the vertical axis 103 between neighboring scaffolds 140, in the opposite direction to the gravity force.

According to some embodiments, each one of the scaffolds 140 comprises two surfaces facing opposite directions along the longitudinal axis 102 of each scaffold 140. According to further embodiments, each one of the scaffolds 140 comprises a first surface 140a and an opposite second surface 140b, facing opposite directions along the longitudinal axis 102 of the scaffold 140.

According to some embodiments, the plurality of openings 131 of the manifolds 130 and 132 are spaced apart from each other along a surface of each manifold, so that at least one opening 131 is positioned at the level of each space volume, in order to enable the liquid flowing therefrom to enter directly into the spaces 145 located between two consecutive scaffolds 140.

According to some embodiments, at least one opening 131 of each of the manifolds 130 and 132 is positioned directly opposing a corresponding space 145 (defined by the space height H1) separating consecutive scaffolds 140 of the scaffold unit 120 within the bioreactor 110, in order to enable direct liquid flow therethrough. According to further embodiments, each one of the openings 131 of the manifolds is positioned directly opposing a corresponding space 145 between two neighboring scaffolds 140, in order to enable direct liquid flow therethrough. In further embodiments, each opening 131 is positioned directly opposing the level of each space 145 separating consecutive scaffolds 140. According to some embodiments, each of the manifolds 130 and 132 comprises at least one top opening 131 which is located above the upper scaffold 146 of the scaffold unit 120, to ensure that the liquid flowing therefrom contacts the upper scaffold 146 and to maintain a desired liquid level within the cell culture bioreactor 110 above the upper scaffold 146.

According to some embodiments, the scaffold unit 120 is disposed within the bioreactor 110, or is coupled to the internal chamber 123 thereof, so that a plurality of opening 131 located on, or embedded within, the first surface 122a (comprising the inlet manifold 130 disposed therein or as an integral part thereof) as disclosed herein above is positioned directly opposing a corresponding space 145 between two neighboring scaffolds 140 of the scaffold unit 120, in order to enable direct liquid flow therethrough. Similarly, a plurality of opening 131 located on, or embedded within, the second surface 124a (comprising the outlet manifold 132) is positioned directly opposing a corresponding space 145 between two neighboring scaffolds 140 of the scaffold unit 120, in order to enable direct liquid flow therethrough.

Advantageously, in some embodiments, the configuration of the cell culture bioreactor 110 comprising the manifolds 130 and 132 and the scaffold unit 120 disposed therein (as disclosed herein), wherein at least one opening 131 is positioned directly opposing a corresponding space 145 (or space height H1) separating consecutive scaffolds 140 of the scaffold unit 120 in order to allow direct liquid flow therethrough within each space 145, can enable uniform and even distribution of the liquid passing between neighboring scaffolds 140, in order to effectively and uniformly support the growth and/or expansion and/or differentiation of cells and/or tissues, cultured on and/or within the plurality of scaffolds 140. Furthermore, the uniform and even distribution of the liquid passing between neighboring scaffolds 140 can reduce or prevent the formation of stresses between the flowing liquid and scaffolds 140 and/or the cells disposed thereon/therein, thereby enabling to evenly and effectively provide the liquid to the scaffolds 140 and/or the cells without harming them in the process.

As used herein, the term “space volume” refers to the volume of each space 145 (defined by a space height H1), spacing between consecutive scaffolds 140 of the scaffold unit 120.

According to some embodiments, each space volume or space 145 evenly separates between consecutive scaffolds 140. According to further embodiments, the volumes or space heights H1 of the spaces 145 separating between consecutive scaffolds 140 within the scaffold unit 120 are even and identical to each other.

According to some embodiments, upon operation of the at least one pump 112, the liquid flows along and/or in parallel to the longitudinal axis 102 between surfaces 140a and 140b of consecutive (i.e., neighboring) scaffolds 140. According to some embodiments, the liquid flows along the width W1 between surfaces 140a and 140b of consecutive scaffolds 140. According to some embodiments, the liquid flows on at least one surface 140a and 140b of the scaffolds 140, within a scaffold's volume, or a combination thereof. According to some embodiments, the liquid flows horizontally in parallel to the longitudinal axis 102 within the space defined by the space height H1, on at least one surface 140a or 140b, of each one of the plurality of scaffolds 140, during the operation of the at least one pump 112. According to some embodiments, the liquid flows along the longitudinal axis 102, such that the liquid contacts at least a part of the first surface 140a and/or the second surface 140b of each scaffold 140, or optionally the liquid contacts the entire first surface 140a and/or the second surface 140b of each scaffold 140.

According to some embodiments, none of the surfaces 140a and 140b of the plurality of scaffolds 140 of the scaffold unit 120 defines an enclosed perimeter across any cross-section of the scaffold unit 120, thus enabling the liquid to pass/flow within the spaces 145 defined by the space height H1 without any disturbances. According to further embodiments, none of the surfaces 140a and 140b of the plurality of scaffolds 140 defines an enclosed perimeter, in parallel to a cross-section of the scaffold structure height SH. According to further embodiments, none of the surfaces 140a and 140b of the plurality of scaffolds 140 defines an enclosed perimeter, in parallel to a cross-section of the width W1. According to further embodiments, none of the surfaces 140a and 140b of the plurality of scaffolds 140 defines an enclosed perimeter, in parallel to a cross-section of the length L1.

According to some embodiments, each one of the scaffolds 140 is planar. According to some embodiments, advantageously, the plurality of scaffolds 140 comprising the plurality of surfaces 140a and 140b are planar and are aligned in the same direction, in parallel to the longitudinal axis 102, thus enabling the liquid to evenly and effectively flow/pass therebetween, within the spaces 145 (each defined by the space height H1).

According to some embodiments, each one of the scaffolds 140 has a scaffold thickness in the range of about 1 mm to about 10 cm. According to further embodiments, the thickness of each of the scaffolds 140 ranges from about 1 mm to about 6 mm. According to further embodiments, the thickness of each of the scaffolds 140 ranges from about 1 cm to about 5 cm. According to some embodiments, when the thickness of each of the scaffold is about 1-6 mm, said thickness of each of the scaffolds 140 is at least 10 times lower compared to the dimension of each of the width W1 and the length L1 of said scaffold 140.

According to some embodiments, each of the two surfaces 140a and 140b of each scaffold 140 has an area of at least 5 cm². According to further embodiments, the area of each of the two surfaces 140a and 140b of each scaffold 140 is above about 10 cm², 50 cm², 100 cm², 200 cm², 500 cm², 1,000 cm², 5,000 cm², or more. Each option represents a different embodiment. According to further embodiments, the area of each of the two surfaces 140a and 140b ranges from about 10 cm² to about 15,000 cm². According to still further embodiments, the area of each of the two surfaces 140a and 140b ranges from about 50 cm² to about 1,000 cm².

According to some embodiments, each one of the scaffolds 140 comprises at least one biocompatible material. According to some embodiments, each one of the scaffolds 140 is made from an edible material. According to certain exemplary embodiments, the edible material is suitable for human consumption. The compositions and texture of the scaffolds of the invention are as is known in the art and as described hereinbelow.

According to some embodiments, the scaffold unit 120 comprises a plurality of extruded, printed, molded, leached, or electro spun scaffolds 140, wherein the scaffolds 140 comprises materials as described hereinbelow. The scaffold unit 120 and/or the plurality of scaffolds 140 can be fabricated without cells by 3D printing, using 3D printing techniques known in the art. Alternately, the scaffold unit 120 and/or the plurality of scaffolds 140 can be fabricated with cells by 3D bioprinting.

According to some embodiments, each one of the scaffolds 140 comprises a porous material. According to some embodiments, each one of the scaffolds 140 is porous. According to some embodiments, each one of the scaffolds 140 is made from one or more porous materials. According to some embodiments, the porous material comprises structured and/or random pores and/or channels. According to further embodiments, the porous material is a structured porous material, a random porous material, or a combination thereof.

According to some embodiments, the porous material comprises pores (i.e., holes or apertures) having a diameter raging from about 2 μm to about 1.5 mm. In some embodiments, the scaffold comprises pores having a diameter ranging from about 20 μm to 1.0 mm. According to still further embodiments, the pores have an average diameter ranging from about 50 μm to about 250 μm.

According to some embodiments, the porous material comprises interconnected pores and/or channels. According to some embodiments, each one of the scaffolds 140 is made from one or more porous materials comprising interconnected pores and/or channels, wherein the pores and/or channels comprise cultured cells and/or tissues disposed therein.

As used herein, the term “channel” refers to a passageway disposed within the porous material along which a fluid may pass. The channels can extend through the porous material or be connected to each other, to form an interconnected channel structure within the porous material.

As used herein, the term “structured porous material” refers to a porous material comprising pores and/or channels, wherein the pores or channels are arranged in an orderly and definable manner with respect to physical characteristics, such as dimensions or orientations.

As used herein, the term “random porous material” refers to a porous material comprising pores and/or channels, wherein the spatial orientation of the cavities (pores or channels), is not controllable.

As used herein, the term “interconnected pores” refers to a porous structure of each one of the scaffolds 140, in which all of its pores are connected to each other to form an inner volume or void space within the material, thus enabling access for liquids from the exterior of each one of the scaffolds 140 to the bulk volume thereof, resulting in fluid communication therethrough.

According to some embodiments, each one of the scaffolds 140 of the scaffold unit 120 is of the same type. According to some embodiments, the scaffold unit 120 comprises different types of scaffolds 140 optionally made from different materials.

According to some embodiments, the scaffold unit 120 comprising the plurality of scaffolds 140 is placed within the cell culture bioreactor 110 so that the surfaces 140a and 140b of consecutive scaffolds 140 are positioned in parallel to the longitudinal axis 102, and optionally in parallel to the bottom surface 111a. According to some embodiments, the scaffold unit 120 is placed within the cell culture bioreactor 110 so that the bottom scaffold 147 thereof is placed on top of the bottom surface 111a of the bioreactor 110, or above an intermediate structure spacing therebetween (e.g., a platform or a holder). According to some embodiments, the scaffold unit 120 is placed within the cell culture bioreactor 110 so that the bottom scaffold 147 thereof is closer to the bottom surface 111a of the bioreactor 110 relative to the upper scaffold 146, wherein both of the bottom scaffold 147 and the upper scaffold 146 are positioned in parallel to the bottom surface 111a. According to some embodiments, the scaffold unit 120 is placed within the cell culture bioreactor 110 so that at least the bottom scaffold 147 thereof is positioned in parallel to the bottom surface 111a of the bioreactor 110, wherein the scaffold unit 120 is coupled to or disposed within a support structure (as disclosed herein above) which is disposed within the bioreactor 110.

According to some embodiments, the at least one pump 112 is configured to direct the flow of the liquid from inlet manifold 130, through the scaffold unit 120, and into the outlet manifold 132, in parallel to the flow direction 101, at a certain flow rate. According to further embodiments, the flow rate of the liquid is controlled to maintain a constant liquid level within the culture bioreactor 110. According to still further embodiments, the flow rate of the liquid is controlled to enable the uniform distribution of the liquid passing through the plurality of spaces 145, each between neighboring scaffolds 140. Without wishing to be bound by any theory or mechanism of action, the controlled flow rate enables/support the cultivation of the cells on and/or within the scaffold.

According to some embodiments, the flow rate of the liquid passing through the plurality of spaces 145 separating consecutive scaffolds 140 of the scaffold unit 120 is controlled by the at least one pump 112, and is above about 0.5 VVD, above about 1 VVD, above about 5 VVD, above about 10 VVD, above about 15 VVD, above about 20 VVD, above about 25 VVD, above about 30 VVD, above about 40 VVD, above about 50 VVD, or more. Each possibility represents a different embodiment.

According to some embodiments, the flow rate of the liquid passing through the plurality of spaces 145 separating consecutive scaffolds 140 of the scaffold unit 120 is in a range of about 0.5-50 VVD. According to further embodiments, the flow rate of the liquid is in the range of about 0.5-10 VVD. According to still further embodiments, the flow rate of the liquid is in the range of about 0.5-6 VVD. According to yet still further embodiments, the flow rate of the liquid is in the range of about 0.5-4 VVD. According to still further embodiments, the flow rate of the liquid is in the range of about 0.5-1.5 VVD.

According to some embodiments, the cultivation system 100 comprises a plurality of pumps 112. According to some embodiments, the cultivation system 100 comprises two or more pumps, wherein a first pump 112A is fluidly coupled to the inlet port 122 and/or the inlet manifold 130, and a second pump 112B is fluidly coupled to the outlet manifold 132 and/or the outlet port 124 (see FIG. 1C). According to some embodiments, the cultivation system 100 comprises a first pump 112A fluidly coupled to the inlet port 122 and/or the inlet manifold 130, a second pump 112B fluidly coupled to the outlet manifold 132 and/or the outlet port 124, and optionally a third pump 112C fluidly coupled directly to the cell culture bioreactor 110. According to some embodiments, the cultivation system 100 comprises three or more pumps, wherein a first pump 112A is fluidly coupled to the inlet port 122 and/or the inlet manifold 130, a second pump 112B is fluidly coupled to the outlet manifold 132 and/or the outlet port 124, and a third pump 112C is fluidly coupled directly to the cell culture bioreactor 110 (see FIGS. 1D-1E).

According to some embodiments, the first pump 112A fluidly coupled to the inlet port 122 and/or the inlet manifold 130 and to the medium reservoir 114. According to some embodiments, the first pump 112A is configured to circulate or flow the liquid from the inlet manifold 130 and into the plurality of spaces 145 separating consecutive scaffolds 140 of the scaffold unit 120 within the bioreactor 110, at an inlet flow rate of above about 0.5 VVD. According to further embodiments, the inlet flow rate is selected from 0.5-10 VVD, optionally 0.5-6 VVD, or alternatively 0.5-1.5 VVD.

According to some embodiments, the second pump 112B is fluidly coupled to the outlet manifold 132 and/or the outlet port 124, and to the medium reservoir 114 or a separation system 116. According to some embodiments, the second pump 112B is configured to collect used liquid from the scaffold unit 120 via the outlet manifold 132, at an outlet flow rate, wherein said outlet flow rate is either identical, lower, or greater than the inlet flow rate. In further embodiments, the outlet flow rate is lower than the inlet flow rate, wherein the outlet flow rate is below about 95%, below about 90%, below about 80%, below about 70%, below about 60%, below about 50%, or less, than the inlet flow rate. In other embodiments, the outlet flow rate is greater than the inlet flow rate, wherein the outlet flow rate is above about 105%, above about 110%, above about 120%, or more, than the inlet flow rate. In some embodiments, the outlet flow rate is above about 0.5 VVD.

According to some embodiments, the third pump 112C is configured to control the liquid level within the cell culture bioreactor 110 in order to maintain a constant liquid level therewithin (see FIGS. 1D-1E). In some embodiments, the third pump 112C is on/off pump, wherein the pump is activated when a liquid contact sensor coupled to the bioreactor 110 detects the presence of liquid, and is deactivated when the contact sensor detects solely air or the absence of liquid within the bioreactor 110. In some embodiments, a liquid level sensor coupled to the bioreactor 110 is adjusted to maintain a desired working liquid level within the cell culture bioreactor 110, according to preprogrammed data or according to real time input from a user.

According to alternative embodiments, the first pump 112A and the second pump 112B can control the liquid level within the cell culture bioreactor 110 by controlling or adjusting the inlet flow rate and/or the outlet flow rate, accordingly. In further such embodiments, the outlet flow rate is adjusted to be lower, identical, or greater than the inlet flow rate, in order to control the liquid level within the cell culture bioreactor 110, or vice versa. In still further such embodiments, the cultivation system 100 does not comprise the third pump 112C (see for example, FIG. 1C).

As used herein, the term “liquid level” refers to an average height level of the liquid within the cell culture bioreactor 110 in parallel to the longitudinal axis 102, wherein the height is measured relative to a certain reference surface. In some embodiments, the reference surface is the upper scaffold 146, and the liquid level within the cell culture bioreactor 110 is measured relative to the upper scaffold 146 of the scaffold unit 120.

According to some embodiments, one or more portions of the scaffold unit 120 are submerged within the liquid flowing/passing within the bioreactor 110 during the activation of the pumps, wherein the liquid reaches the desired liquid level above the upper scaffold 146 thereof. According to further embodiments, the entire scaffold unit 120 is submerged within the liquid flowing/passing within the bioreactor 110, wherein the liquid reaches the desired liquid level above the upper scaffold 146 thereof.

According to some embodiments, third pump 112C is fluidly coupled to the cell culture bioreactor 110 and to the medium reservoir 114 (see FIG. 1D), optionally via one or more fluid conduit(s) 105, and is configured to transfer excess liquid from the bioreactor 110 directly into the medium reservoir 114, in order to maintain a desired liquid level within the cell culture bioreactor 110 above the upper scaffold 146 of the scaffold unit 120.

According to some embodiments, the desired liquid level within the cell culture bioreactor 110, optionally resulting from the operation of the third pump 112C, is at least about 0.1 mm above the upper scaffold 146 of the scaffold unit 120. In further embodiments, the desired liquid level within the cell culture bioreactor 110 is at least about 1 mm, at least about 5 mm, at least about 6 mm, at least about 10 mm, or more, above the upper scaffold 146 of the scaffold unit 120. Each possibility represents a different embodiment. In a certain embodiment, the desired liquid level within the cell culture bioreactor 110 is at least about 6 mm above the upper scaffold 146 of the scaffold unit 120.

According to some embodiments, the desired liquid level within the cell culture bioreactor 110 ranges from about 0.1 mm to about 100 mm above the upper scaffold 146 of the scaffold unit 120. In further embodiments, the desired liquid level within the cell culture bioreactor 110 ranges from about 0.1 mm to about 30 mm above the upper scaffold 146 of the scaffold unit 120. In still further embodiments, the desired liquid level within the cell culture bioreactor 110 ranges from about 0.1 mm to about 1 mm, about 1 mm to about 5 mm, about 5 mm to about 10 mm, about 10 mm to about 20 mm, or about 20 mm to about 30 mm above the upper scaffold 146 of the scaffold unit 120. Each possibility represents a different embodiment. In some embodiments, the desired liquid level within the cell culture bioreactor 110 ranges from about 6 mm to about 30 mm above the upper scaffold 146 of the scaffold unit 120. In further embodiments, the desired liquid level within the cell culture bioreactor 110 ranges from about 1 mm to about 10 mm above the upper scaffold 146 of the scaffold unit 120. In an exemplary embodiment, the desired liquid level within the cell culture bioreactor 110 is about 6 mm above the upper scaffold 146 of the scaffold unit 120.

According to some embodiments, the desired liquid level within the cell culture bioreactor 110 (above the upper scaffold 146 of the scaffold unit 120) corresponds or is identical to the space height H1 of each space 145 formed between neighboring scaffolds. In further such embodiments, the desired liquid level within the cell culture bioreactor 110 above the upper scaffold 146 of the scaffold unit 120 and each space height H1 ranges from about 0.1 mm to about 30 mm, or preferably from about 0.1 mm to about 10 mm. In an exemplary embodiment, the desired liquid level within the cell culture bioreactor 110 above the upper scaffold 146 of the scaffold unit 120 and each space height H1 is about 6 mm. In is contemplated, in some embodiments, that matching the liquid level within the cell culture bioreactor 110 (above the upper scaffold 146 of the scaffold unit 120) to the space height H1 of each spaces 145 formed between neighboring scaffolds, enables to form the advantageous uniform and effective liquid flow distribution between consecutive scaffolds 140, as disclosed herein above.

Advantageously, in some embodiments, the first pump 112A, second pump 112B, and optionally the third pump 112C enable to control the flow rate of the liquid flowing through the scaffold unit 120 and further enable to maintain a constant liquid level within the culture bioreactor 110, thereby ensuring the uniform and effective distribution of the liquid passing through the plurality of spaces 145. As a result of the uniform and effective liquid flow distribution between consecutive scaffolds 140, the liquid can effectively support the growth and/or expansion and/or differentiation of cells and/or tissues cultured on and/or within the scaffolds 140. Furthermore, the flow rate of the liquid passing through the plurality of scaffolds 140 is controlled in order to enable an optimal distribution of the liquid parameters (e.g., dissolved oxygen content) along the surfaces of neighboring scaffolds 140, thus supporting optimal and enhanced cell cultivation thereon. In addition, the uniform and effective liquid flow distribution between consecutive scaffolds 140 can reduce or prevent the formation of stresses between the flowing liquid and the scaffolds 140 and/or the cells disposed thereon/therein, thereby enabling to evenly and effectively provide the liquid to the scaffolds 140 and/or the cells without harming them in the process.

According to some embodiments, the cultivation system 100 further comprises at least one sensor 152 (see for example, FIG. 1A) coupled thereto, or optionally a plurality of sensors 152. According to some embodiments, the at least one sensor 152 is a liquid level sensor coupled to the culture bioreactor 110 and is configured to measure the liquid level therein. According to other embodiments, the at least one sensor 152 is a liquid contact sensor configured to detect the presence of liquid within the bioreactor 110, or the absence thereof. According to some embodiments, the at least one sensor 152 is coupled to at least one surface of the internal chamber 123 of the bioreactor 110, to the medium reservoir 114, to the outlet port 124, to a fluid conduit 105 which is extending from the outlet port 124 and is fluidly coupled thereto, or a combination thereof. Each possibility represents a different embodiment.

According to some embodiments, the system 100 further comprises a control unit 154 (see for example, FIG. 1A). According to further embodiments, the control unit 154 is in operative communication (through a wired or wireless connection) with one or more of: the at least one sensor 152, the medium reservoir 114, the at least one pump 112, or a combination thereof. According to further embodiments, the control unit 154 is in operative communication with one or more of: the first pump 112A, second pump 112B, and the third pump 112C (see for example, FIG. 1E).

According to some embodiments, the control unit 154 is configured to receive data from the at least one sensor 152, wherein the data is indicative of the presence or absence of liquid within the bioreactor 110 and/or the medium reservoir 114, and to adjust the activation and operation of the at least one pump 112 accordingly, thereby adjusting the desired liquid flow rate and liquid level within the bioreactor 110.

According to other embodiments, the control unit 154 is configured to receive measurements of the liquid level within the bioreactor 110 and/or the medium reservoir 114, and adjust the liquid level and/or the flow rate therethrough, based on said measurements. According to further such embodiments, the control unit 154 is configured to receive measurements of the liquid level within the internal chamber 123 from the at least one sensor 152, and to adjust the liquid level and/or the flow rate thereof based on the measurements, by adjusting the operation of the at least one pump 112, and specifically by adjusting the operation of the first pump 112A, second pump 112B, and the third pump 112C.

According to some embodiments, the control unit 154 is configured to receive measurements of at least one medium parameter within the bioreactor 110 and/or the medium reservoir 114 and adjust or modify said at least one medium parameter if needed, wherein the medium parameter is selected from the group consisting of: temperature, pH, dissolved oxygen content, concentration of one or more nutrients, concentration of one or more waste products, and any combination thereof. Each possibility represents a separate embodiment. For example, the control unit 154 can receive temperature measurements and adjust the temperature within the medium reservoir 114 and/or the bioreactor 110, by actuating or adjusting a suitable temperature control apparatus. According to further embodiments, the control unit 154 is configured to receive measurements of a plurality of medium parameters within the bioreactor 110 and/or the medium reservoir 114, and adjust or modify said plurality of medium parameters simultaneously, wherein the plurality of medium parameters can include for example, temperature, pH, and dissolved oxygen content.

As used herein, the term “adjust” in the context of adjusting the at least one medium parameter, refers to the control unit 154 actuating one or more suitable apparatus(s) (e.g., a temperature apparatus, separation system 116, etc.) according to the measurements of at least one medium parameter, as disclosed herein above.

According to some embodiments, the control unit 154 comprises a programmable logic controller (PLC) or a programmable controller, as is typically known in the art. According to some embodiments, the PLC comprises at least one input/output component, at least one processor, a plurality of memory modules and kernel logic, or other functional standard components.

According to some embodiments, the liquid comprises a cell culture medium, configured to support the cultivation of cells and/or tissues. According to some embodiments, the medium comprises at least one material selected from the group consisting of water, salts, nutrients, minerals, dissolved oxygen, vitamins, amino acids, nucleic acids, proteins (such as cytokines and growth factors), hormones, serum, trace elements, or any combination thereof. According to some embodiments, the medium as used herein refers to a liquid substance which is required for cell proliferation and/or promote cell growth and/or expansion and or promote cell differentiation.

According to some embodiments, the system 100 further comprises at least one medium reservoir 114 which is fluidly coupled to the at least one bioreactor 110, wherein the medium reservoir 114 is configured for supplying cell cultivation medium thereto. According to some embodiments, the medium reservoir 114 is further fluidly coupled to the third pump 112C. According to some embodiments, the medium reservoir 114 is configured to receive and/or contain the medium therein. According to further embodiments, the system 100 comprises a plurality of medium reservoirs 114. According to some embodiments, the medium reservoir 114 comprises a mixed/stirred container, configured to mix or stir the medium disposed therein, in order to maintain the homogeneity of the medium. According to some embodiments, the medium reservoir 114 comprises a mixing or stirring apparatus disposed therein, which is configured to mix/stir the medium disposed therein, thereby forming a homogenous medium. According to some embodiments, the medium reservoir 114 comprise a wave bioreactor, a stirred bioreactor, or a combination thereof. Each possibility represents a different embodiment.

According to some embodiments, the medium reservoir 114 comprises at least one sensor coupled thereto (see for example, FIG. 1C), wherein the sensor is configured to measure at least one medium parameter within the medium, and wherein the medium parameter is selected from the group consisting of: temperature, pH, dissolved oxygen, concentration of one or more nutrients, concentration of one or more waste products, and any combination thereof. Each possibility represents a separate embodiment. In some embodiments, the at least one sensor configured to measure at least one medium parameter is identical to sensor 152 as disclosed herein above, or wherein the sensor is another type of sensor known in the art. In some embodiments, the at least one sensor is configured to transfer data indicative of the at least one medium parameter to the control unit 154, and the control unit 154 is configured to adjust or modify said at least one medium parameter if needed, according to pre-programmed medium requirements and/or according to user demand.

According to some embodiments, the medium reservoir 114 is operated in a perfusion mode, wherein spent or used medium enters thereto from the bioreactor 110, the medium parameters are measured and adjusted as disclosed herein above in order to transform the spent medium entering the medium reservoir 114 into treated medium, and then said treated medium is circulated back into the bioreactor 110, and so on. In alternative embodiments, spent medium entering into the medium reservoir 114 is replaced with new or fresh medium which is circulated back into the bioreactor 110.

According to some embodiments, the cultivation system 100 comprises at least two cell culture bioreactors 110, fluidly coupled in parallel to each other, and optionally to the at least one medium reservoir 114, as illustrated at FIG. 1B.

According to some embodiments, the system 100 further comprises at least one separation system 116 (see FIG. 1E). According to further embodiments, the separation system 116 is configured to receive spent medium exiting the bioreactor 110 and remove waste products residing therein (such as ammonia and lactic acid), thereby providing a treated cultivation medium which is transferred into the medium reservoir 114 and/or into the bioreactor 110. According to some embodiments, the separation system 116 is configured to remove waste products residing within the medium flowing therethrough, utilizing one or more separation methods or techniques selected from the group consisting of; size exclusion, electrophoresis, ion exchange, other separation methods known in the art, or a combination thereof. Each possibility represents a different embodiment.

According to some embodiments, the separation system 116 comprises a treatment vessel comprising therein appliances desired for performing the one or more separation methods or techniques as disclosed above. According to further embodiments, the separation system 116 comprises a plurality of treatment vessels fluidly coupled to one another, wherein each one comprises one or more appliances desired for at least one separation method therein.

According to some embodiments, the separation system 116 is fluidly coupled to the bioreactor 110 and to medium reservoir 114. According to some embodiments, the separation system 116 is disposed within the medium reservoir 114. According to other embodiments, the separation system 116 is separate from the medium reservoir 114.

According to some optional embodiments, the separation system 116 comprises at least one sensor (e.g., sensor 152) which is configured to measure in the medium at least one medium parameter as disclosed herein above.

According to some embodiments, the separation system 116 comprises a dialysis system (not shown). The dialysis system typically comprises a dialyzer, a fresh dialysate reservoir and a used dialysate reservoir (not shown).

According to some embodiments, the bioreactor 110 further comprises a temperature control apparatus (not shown) which is configured to control the temperature therein, and is optionally disposed within the bioreactor 110, in order to provide a temperature which is optimal for cell culture and/or growth. According to some embodiments, the bioreactor 110 is disposed within a temperature control vessel configured to control the temperature within the bioreactor 110 (e.g., a heating blanket or jacket or incubator).

According to some embodiments, the system 100 further comprises a delivery system configured to deliver the medium from the medium reservoir 114 into the at least one cell culture bioreactor 110 via the inlet manifold 130, and collecting used or spent medium from said cell culture bioreactor 110 via the outlet manifold 132. The delivery system can include the at least one the pump 112, additional pump(s), other suitable appliances (e.g., tubes, pipes, liquid lines, such as the fluid conduits 105), and combinations thereof. According to some embodiments, cultivation system 100 comprises a plurality of pumps 112 (e.g., the first pump 112A, second pump 112B, and the third pump 112C). The cultivation system 100 can include additional types of pumps, optionally different from pump 112.

According to some embodiments, the at least one pump 112 comprises one or more positive displacement pumps. According to some embodiments, the at least one pump 112 comprises one or more peristaltic pumps. According to some embodiments, the delivery system is further configured to deliver the used or spent medium to the separation system 116 and/or into medium reservoir 114.

According to some embodiments, the cultivation system 100 comprises a plurality of pumps 112, wherein said plurality of pumps 112 can be identical to each other, or include different types of pumps for various uses in the system. According to some embodiments, the cultivation system 100 comprises a plurality of pumps 112, wherein at least one pump 112 can be used to enable the adjustment of medium parameters within the system (e.g., pH and the like), flow medium into and/or from the system, and other relevant options.

According to some embodiments, the at least one pump 112 comprises one or more dual head pumps. According to some embodiments, the at least one pump 112 is selected from a peristaltic pump, diaphragm pump, dual head pump, combinations thereof, or any other known pump in the art. Each possibility represents a separate embodiment of the present invention. According to some embodiments, one or more of the first pump 112A, second pump 112B, and the third pump 112C are selected from a peristaltic pump, diaphragm pump, dual head pump, combinations thereof, or any other known pump in the art. Each possibility represents a separate embodiment of the present invention. According to a specific embodiment, each one of the first pump 112A, second pump 112B, and the third pump 112C are peristaltic pumps.

According to some embodiments, the at least one pump 112 is configured to pump in and/or out the medium to and/or from the bioreactor 110. According to some embodiments, the at least one pump 112 is configured to deliver the medium from the medium reservoir 114 into the at least one cell culture bioreactor 110 via the inlet manifold 130, to collect used medium from the cell culture bioreactor 110 via the outlet manifold 132, and optionally to circulate the medium within the system 100. According to further embodiments, the at least one pump 112 is configured to deliver the medium from the medium reservoir 114 into the at least one cell culture bioreactor 110 via the inlet manifold 130, and to collect used medium from the cell culture bioreactor 110 via the outlet manifold 132, at the same rate. According to other embodiments, the first pump 112A is configured to deliver the medium from the medium reservoir 114 into the at least one cell culture bioreactor 110 via the inlet manifold 130 at the inlet flow rate, and the second pump 112B is configured to collect used medium from the cell culture bioreactor 110 via the outlet manifold 132 at the outlet flow rate, wherein the outlet flow rate is identical or is lower than the inlet flow rate, as disclosed herein above.

According to some embodiments, the system 100 comprises one or more sensors 152 for measuring in the medium at least one medium parameter selected from: temperature, pH, dissolved oxygen content, concentration of one or more nutrients, concentration of one or more waste products, and any combination thereof. Each possibility represents a separate embodiment. According to further embodiments, the one or more sensors 152 are located in at least one of the medium reservoir 114, the bioreactor 110, the separation system 116, the delivery system, and combinations thereof. According to still further embodiments, the one or more sensors 152 are located at the outlet port 124 of the bioreactor 110 and/or at a surface of the internal chamber 123 and/or at the medium reservoir 114. According to some embodiments, the control unit 154 is in operative communication with at least one of the sensors 152, the separation system 116, the delivery system, the at least one pump 112, the medium reservoir 114, the bioreactor 110, and combinations thereof. Each possibility represents a separate embodiment. According to further embodiments, the control unit 154 is configured to receive measurements of the at least one parameter and to adjust it based on the measurements thereof.

According to some optional embodiments, the bioreactor 110 and/or the medium reservoir 114 comprises an electric component or an actuator, configured to generate the movement of the liquid flowing therethrough, optionally by providing external movement to the bioreactor 110 and/or the medium reservoir 114, accordingly. According to further embodiments, the movement is selected from vibrating, rotating, tilting, shaking, rocking, or any other form of movement known in the art. Each possibility represents a separate embodiment of the present invention. The movement can enable to achieve optimal values of various medium parameters (e.g., dissolved oxygen content) within the bioreactor 110. According to some embodiments, the scaffold unit 120 comprising the plurality of scaffolds 140 is directly or indirectly attached to at least one surface of the internal chamber 123, in order to prevent its movement during the movement of the liquid.

According to some embodiments, in order to achieve optimal values of dissolved oxygen content within the bioreactor 110, various gasses are flowed within the bioreactor 110 (e.g., oxygen and/or air). According to some embodiments, in order to control pH within the system 100, 5% CO₂ (w/w) is flowing into the bioreactor 110. According to some embodiments, in order to control pH within the medium reservoir 114, the liquid flowing thereto receives CO₂ gas for lowering the pH, or one or more base molecules for increasing the pH.

The term “actuator”, as used herein, refers to any powered actuator known in the art for providing rotational motion, such as an electric motor, a solenoid, and the like.

According to some optional embodiments, the system 100 further comprises a cell trap located between the outlet port 124 of the bioreactor 110 and an inlet of the medium reservoir 114. According to further embodiments, the delivery system further comprises the cell trap located between the outlet port 124 of the bioreactor 110 and an inlet of the medium reservoir 114.

The presence of circulating gas bubbles within the system 100 may damage at least one of the various appliances of the system (e.g., the pump 112), the cells cultured on the scaffolds 140, and may cause measurement errors of the at least one medium parameter. According to some optional embodiments, the system 100 further comprises at least one microfluidic bubble trap (termed “debubbler”), configured to remove/expel gas bubbles from the system 100.

According to some embodiments, the system 100 further comprises an oxygenator (not shown) configured to continuously provide oxygen to the medium. According to some embodiments, the oxygenator is configured to continuously provide oxygen to the medium through aeration. According to some embodiments, the oxygenator is fluidly coupled to the medium reservoir 114. According to some embodiments, the oxygenator (e.g., a sparger) is disposed within the medium reservoir 114 or is an integral part thereof.

Advantageously, the system 100 of the present invention is suitable for culturing various types of cells and/or products thereof, for a wide range of uses. According to certain exemplary embodiments, the cultured cells are non-human-animal cells, and the systems are used to produce culture food products. Cells to be used according to these embodiments are as known in the art and as described hereinbelow.

According to another aspect, there is provided a method for producing at least one scaffold unit 120 comprising a plurality of scaffolds 140 comprising cultured cells and/or tissues thereon and/or therewithin, comprising the steps of:
a. providing at least one scaffold unit 120 comprising the plurality of scaffolds 140, as disclosed herein above;
b. seeding cells on at least part of a surface or within each scaffold of the plurality of scaffolds 140;
c. optionally placing said scaffold unit 120 comprising the plurality of scaffolds 140 under conditions enabling adherence of the seeded cells thereto, until said cells are at least partially covering a surface 140a and/or 140b of each of the scaffolds 140 or until the cells are covering at least a part of the pores and/or channels of each of the scaffolds 140;
d. providing the system 100 as disclosed herein above, and placing the scaffold unit 120 comprising the plurality of scaffolds 140 having at least part of each scaffold seeded with said cells in the cell culture bioreactor 110, wherein the scaffold unit 120 is in a configuration as disclosed herein above, specifically wherein each scaffold 140 is separated from its neighboring scaffolds to form spaces 145 along which a liquid may pass, said cell culture bioreactor 110 further comprising an inlet manifold 130 and an outlet manifold 132 each configured to direct the flow of the liquid through the plurality of scaffolds 140, wherein the manifolds are fluidly connected to at least one pump 112 configured to control the flow rate of said liquid through the plurality of scaffolds 140;
e. operating the at least one pump 112 to deliver cell culture medium from the inlet manifold 130 along said spaces 145 of the scaffold unit 120 into the outlet manifold 132, and
f. cultivating the cells on and/or within the plurality of scaffolds, thereby producing a plurality of scaffolds comprising cultured cells and/or tissues.

According to some embodiments, the method further comprises a step of removing the scaffold unit 120 comprising the cultured cells and/or tissues from the bioreactor 110.

According to some embodiments, step (b) of seeding the cells comprises seeding cells on at least some of the scaffolds 140. According to some embodiments, step (b) of seeding the cells comprises seeding cells on one or two of the surfaces 140a and 140b of each scaffold 140.

According to alternative embodiments, step (b) of seeding the cells comprises administering the cells directly into each scaffold 140, or into the spaces 145 separating neighboring scaffolds 140. According to some embodiments, step (b) of seeding the cells comprises administering the cells directly into each scaffold 140, by performing one or more of the following actions: injecting, dripping, pipetting, and the like. Each possibility represents a different embodiment. According to further embodiments, step (b) of seeding the cells comprises injecting the cells directly into at least part of the pores or channels of each scaffold 140. According to some embodiments, step (b) of seeding the cells comprises administering the cells into the spaces 145 separating neighboring scaffolds 140, wherein the cells are administered within a suspension medium comprising the cells, wherein the suspension medium is dispersed or sprayed into said spaces 145 and contact and attach into the surfaces of each scaffold 140.

According to some embodiments, step (c) of placing the scaffold unit 120 comprising the plurality of scaffolds 140 under conditions enabling adherence of the seeded cells thereto is performed within the cell culture bioreactor 110 or outside thereof. According to some embodiments, step (c) is optional.

According to some embodiments, if step (c) was performed within the cell culture bioreactor 110, then step (d) is combined with step (c).

According to some embodiments, the inlet manifold 130 and the outlet manifold 132 each comprises a plurality of openings 131 spaced from each other along a surface of each manifold and extending therethrough, wherein step (d) comprises placing the scaffold unit 120 in the cell culture bioreactor 110 such that at least one opening 131 is positioned directly opposing a corresponding space 145 separating consecutive scaffolds 140 of the scaffold unit 120, in order to enable direct liquid flow therethrough.

According to some embodiments, step (e) comprises delivering the cell culture medium, by operating the at least one pump 112, from the plurality of openings 131 of the inlet manifold 130, respectively into each space 145 separating consecutive scaffolds 140 of the scaffold unit 120, and then into the plurality of openings 131 of the outlet manifold 132. According to some embodiments, step (e) comprises delivering the cell culture medium from at least one top opening 131 of the inlet manifold 130, above the upper scaffold 146 of the scaffold unit 120, and into at least one top opening 131 of the outlet manifold 132.

According to some embodiments, step (e) comprises delivering the cell culture medium from the plurality of openings 131 of the outlet manifold 132 into the plurality of openings 131 of the inlet manifold 130, optionally via one or more intermediate system components or apparatuses, thereby circulating the liquid within the system 100. According to some embodiments, step (e) comprises delivering the cell culture medium from (i) the plurality of openings 131 of the outlet manifold 132 into medium reservoir 114, and (ii) from medium reservoir 114 into the plurality of openings 131 of the inlet manifold 130.

According to some embodiments, the system 100 comprises a plurality of pumps, wherein during step (e) a first pump 112A is delivering the medium (optionally from the medium reservoir 114) into the at least one cell culture bioreactor 110 via the inlet manifold 130 at the inlet flow rate, a second pump 112B is collecting used medium from the cell culture bioreactor 110 via the outlet manifold 132 at the outlet flow rate, and optionally a third pump 112C is fluidly coupled to the bioreactor 110, wherein one or more of the first pump 112A, the second pump 112B, and optionally the third pump 112C controls the liquid level within the cell culture bioreactor 110. According to some embodiments, the inlet flow rate is above about 0.5 VVD. According to some embodiments, the desired liquid level within the cell culture bioreactor 110, optionally resulting from the operation of the third pump 112C, is at least about 0.1 mm above the upper scaffold 146 of the scaffold unit 120.

According to some embodiments, step (e) further comprises flowing spent medium exiting the bioreactor 110 via the outlet manifold 132 into at least one of a separation system 116, a cell trap, or a combination thereof. Each possibility represents a different embodiment. According to further embodiments, the spent medium is treated within the separation system 116 and fresh or treated medium is circulated again into the bioreactor 110 via the medium reservoir 114.

According to some embodiments, step (f) comprises cultivating the cells on and/or within the plurality of scaffolds 140 to reach at least one pre-set parameter selected from the group consisting of a desired tissue mass; nutrient uptake rate; oxygen uptake rate; waste production rate; and any combination thereof, thereby producing a plurality of scaffolds comprising cultured cells and/or tissues thereon and/or therewithin. According to some embodiments, the nutrient is selected from the group consisting of glucose and glutamine. According to some embodiments, the waste product is selected from the group consisting of ammonium and lactate. According to some embodiments, the at least one pre-set parameter is measured by the at least one sensor 152 or other suitable sensor(s), which sends parameter data to the control unit 154, as disclosed herein above.

According to some embodiments, the liquid is a cell culture medium. According to some embodiments, the method is performed under aseptic conditions. According to some embodiments, the cells are non-human-animal cells.

According to some embodiments, the method is configured for producing a cultured food product. According to further embodiments, the cultured food product is cultured meat.

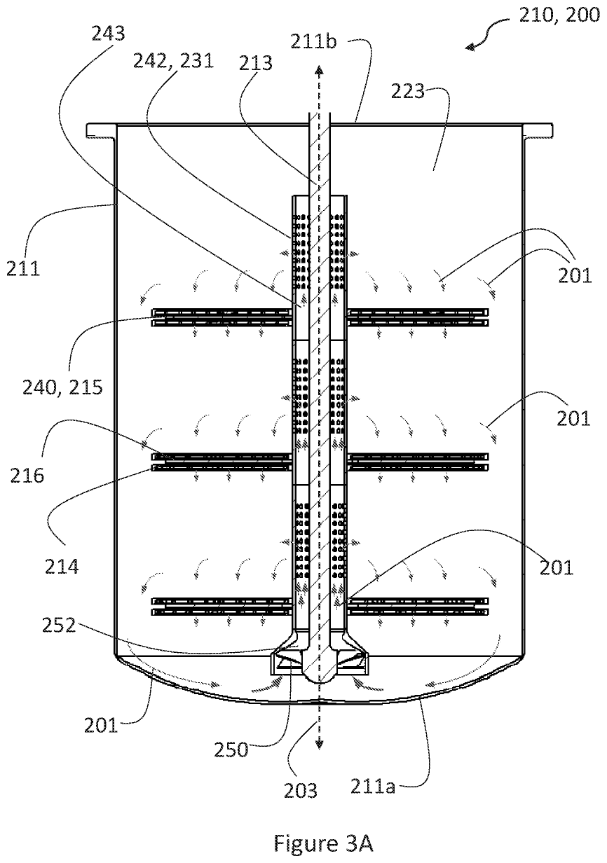

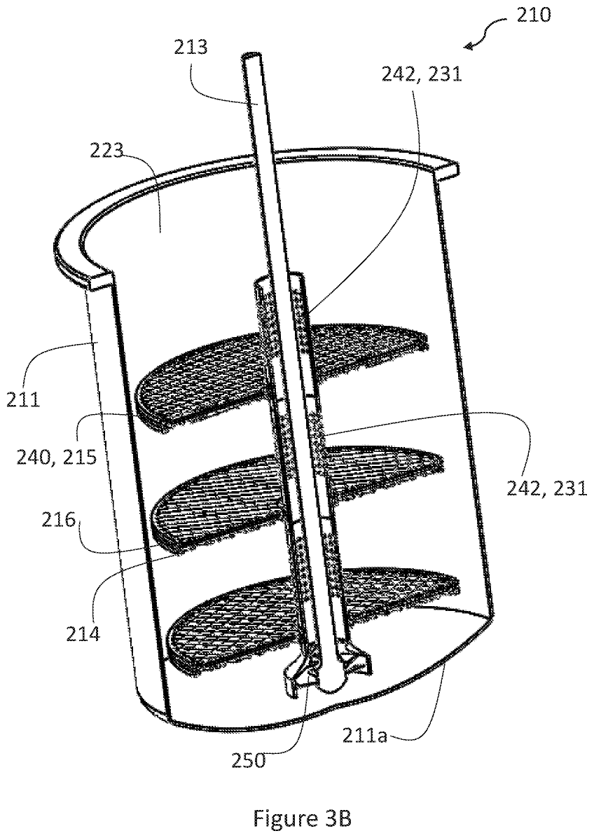

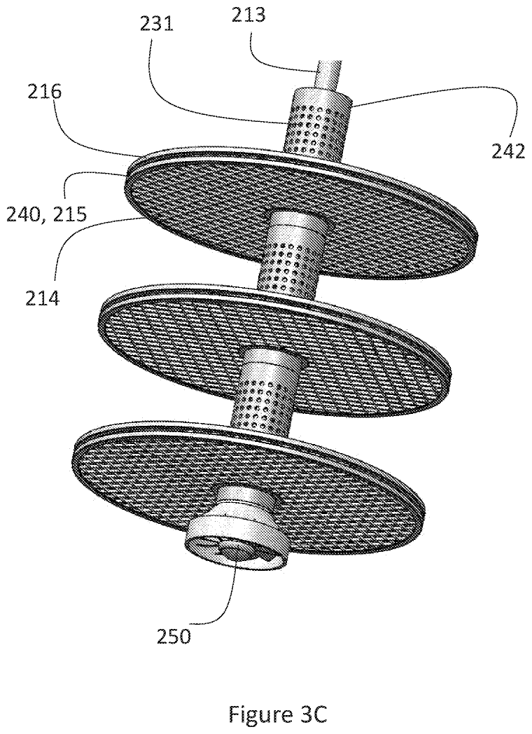

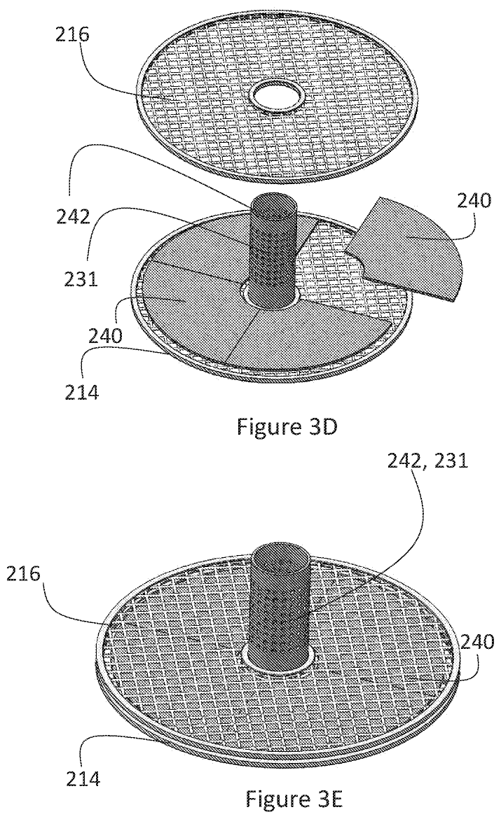

Reference is now made to FIGS. 3A-3E. FIG. 3A is a cross sectional view of a scheme of a bioreactor 210, according to some embodiments. FIG. 3B is a cross sectional view in perspective of bioreactor 210, according to some embodiments. FIGS. 3C-3E are views in perspective of components of bioreactor 210, according to some embodiments.

According to another aspect, there is provided a cultivation system 200 configured to deliver a liquid to a plurality of scaffolds, wherein the liquid may comprise cells to be seeded on and/or within the scaffolds, wherein the liquid is a medium supporting the growth and/or proliferation and/or differentiation and/or expansion of a population of cells already seeded on the scaffolds. According to some embodiments, the system 200 is configured to deliver said liquid to said population of cultured cells and/or tissues, in order to enable the production of cultured cells and/or tissues, particularly cultured food products.

According to some embodiments, the cultivation system 200 is different from to the cultivation system 100 as disclosed herein above. According to alternative embodiments, the cultivation system 200 is identical to the cultivation system 100 as disclosed herein above, except for the configuration of the bioreactor as elaborated herein below.

According to some embodiments, the cultivation system 200 comprises a bioreactor 210, wherein said bioreactor 210 is a single vessel that is configured to deliver a liquid to a plurality of scaffolds, optionally comprising a population of cultured cells and/or tissues, as one unit, optionally without utilizing additional appliances. According to further embodiments, bioreactor 210 is configured to deliver said liquid to said plurality of scaffolds in order to seed cells on at least part of the plurality of scaffolds and/or enable the large-scale production of cultured food products at a cost-effective manner. According to some embodiments, the bioreactor 210 is configured to agitate such that the liquid is circulated by suction and delivered to the population of cultured cells and/or tissues, in order to enable the production of cultured cells and/or tissues, particularly cultured food products.

According to some embodiments, the bioreactor 210 can be operated in a mode selected from a batch, fed batch or perfusion mode(s). According to some embodiments, the bioreactor 210 is a stirred bioreactor. According to some embodiments, the bioreactor 210 is a stirred bioreactor, operated in a mode selected from a batch, fed batch or perfusion mode(s).

According to certain embodiments, the liquid is a cell-suspension medium comprising a plurality of cells to be seeded on the scaffolds, wherein the cell-suspension medium is configured to seed the cells on at least part of the plurality of scaffolds.

According to some embodiments, the liquid is a cell culture medium which supports the cultivation of cells and/or tissues, as disclosed hereinabove.

According to some embodiments, the cell culture medium is identical to the cell-suspension medium. According to some embodiments, the cell culture medium is different from the cell-suspension medium.

According to some embodiments, the liquid comprises an aqueous solution comprising at least one buffer for washing the scaffolds and/or the internal volume of the bioreactor 210.

According to some embodiments, the liquid comprises at least one of the cell-suspension medium, the cell culture medium, the buffer, and combinations thereof. Each possibility represents a different embodiment.

According to some embodiments, bioreactor 210 comprises at least one bioreactor wall 211 defining an internal chamber 223, and is positioned substantially perpendicularly to a bottom surface 211a and an upper surface 211b. According to further embodiments, the at least one bioreactor wall 211 is extending from the bottom surface 211a to the upper surface 211b, in parallel to a vertical axis 203. According to some embodiments, bioreactor 210 comprises at least one main tube 213, positioned along the vertical axis 203, and extending perpendicularly from the upper surface 211b towards the internal chamber 223. According to further embodiments, the at least one main tube 213 extends through the upper surface 211b.

According to some embodiments, the bioreactor 210 further comprises a plurality of cultivation trays 215 disposed within the internal chamber 223, wherein said plurality of cultivation trays 215 are directly or indirectly coupled to the at least one main tube 213.

According to some embodiments, the plurality of cultivation trays 215 are attached to at least one supportive element 242 and are spaced from each other therealong, wherein said at least one supportive element 242 is attached to the main tube 213. According to further embodiments, the plurality of cultivation trays 215 extends radially from the at least one supportive element 242, wherein the radial direction is perpendicular to the vertical axis 203. According to further embodiments, the at least one supportive element 242 is shaped as a hollow tube or cylinder which surrounds or encompasses the main tube 213, thus defining an inner supportive element space 243 between the main tube 213 and the supportive element 242, wherein the liquid can pass/flow through (e.g., a cell-suspension medium and/or a cell culture medium). According to some embodiments, the at least one supportive element 242 comprises a plurality of openings 231 extending therethrough and are spaced from each other along a circumference thereof, each configured to allow liquid flow therethrough.

According to some embodiments, the plurality of cultivation trays 215 extends radially from a corresponding plurality of supportive elements 242, wherein each supportive element 242 is attached to at least one cultivation tray 215, wherein the radial direction is perpendicular to the vertical axis 203 (illustrated at FIGS. 3A-3E). According to further embodiments, the plurality of supportive elements 242 surrounds or encompasses the main tube 213. According to further embodiments, each supportive element 242 comprises a plurality of openings 231 spaced from each other along a circumference thereof, each configured to allow liquid flow therethrough. According to further embodiments, the plurality of supportive elements 242 are fluidly coupled to one another, wherein each supportive element 242 is shaped as a hollow tube or cylinder, thus defining the inner supportive element space 243 therebetween, wherein the liquid can pass/flow through (e.g., the cell-suspension medium and/or the cell culture medium).

According to some embodiments, each cultivation tray 215 comprises a bottom tray 214 and an upper tray 216, supporting at least one scaffold 240 disposed therebetween. According to further embodiments, said scaffold 240 is disposed between the bottom tray 214 and the upper tray 216. According to some embodiments, each one of the bottom tray 214 and the upper tray 216 comprise a mesh and/or a porous structure, in order to enable the liquid to enter/pass/flow therethrough and to contact the at least one scaffold 240 residing therebetween.

According to some embodiments, the plurality of cultivation trays 215 comprises a plurality of scaffolds 240, wherein each cultivation tray 215 is configured to enable the liquid (e.g., the cell-suspension medium and/or the cell culture medium) to flow/pass therethrough and/or therealong and optionally between neighboring or consecutive scaffolds 240, in order to support the growth and/or expansion and/or differentiation of cells and/or tissues, cultured on the plurality of scaffolds 240.

According to some embodiments, the plurality of openings 231 are located above each respective cultivation tray 215 along the vertical axis 203, in order to enable optimal liquid passage from the openings 231 towards the scaffolds 240, facilitated by the gravitational force.

According to some embodiments, the plurality of supportive elements 242 are configured to space between the plurality of cultivation trays 215, and to provide support thereto, in order to enable optimal liquid (e.g., a cell-suspension medium and/or the cell culture medium) passage between neighboring or consecutive cultivation trays 215, along at least one surface of each cultivation tray 215, through the plurality of cultivation trays 215, and combinations thereof. Each possibility represents another embodiment.

According to some embodiments, each scaffold 240 is identical to each scaffold 140, as disclosed herein above. According to some embodiments, scaffold 240 is made from the same materials and has the same properties as scaffold 140, as disclosed hereinabove.

According to some embodiments, each cultivation tray 215 is planar. According to some embodiments, advantageously, the plurality of cultivation trays 215 comprising the plurality of scaffolds 240 are planar and are aligned in the same direction in parallel to each other and perpendicularly to the vertical axis 203, thus enabling the liquid to evenly flow/pass therebetween.

According to some embodiments, each cultivation tray 215 is disc shaped. However, it is to be understood that the cultivation tray 215 fulfills the same function when otherwise shaped, as a sphere, ring, ovoid, ellipsoid, square, or any other polyhedron. Each possibility represents a separate embodiment of the present invention.

According to some embodiments, the shape of the plurality of scaffolds 240 corresponds to the shape of each cultivation tray 215. According to some embodiments, the plurality of scaffolds 240 are shaped as at least a portion of a disc or a circle (e.g., one quarter of a disc or circle, FIG. 3D).

According to some embodiments, bioreactor 210 further comprises at least one mixing apparatus 250, attached to the main tube 213, and optionally is located in the vicinity of the bottom surface 211a (illustrated at FIGS. 3A-3B). According to some embodiments, the mixing apparatus 250 is spaced from the bottom surface 211a, so that it does not directly contacts the bottom surface 211a, thus enabling liquid passage therebetween. According to some embodiments, the mixing apparatus 250 is selected from an impeller, mixer, propeller, blender, other suitable mixing apparatus, or a combination thereof. Each possibility represents a different embodiment. According to further embodiments, the mixing apparatus 250 is an impeller 250.

As used herein, the term “vicinity” refers to a distance within a radius of less than about 200 mm of a given three-dimensional (3D) space. According to some embodiments, the term “vicinity” refers to a distance within a radius of less than about 50 mm, preferably less than about 10 mm, more preferably less than about 1 mm, or even more preferably less than about 0.1 mm of a given 3D space. Each possibility represents a separate embodiment of the present invention.

According to some embodiments, the mixing apparatus 250 (e.g., impeller 250) is configured to circulate the liquid within the bioreactor 210, in order to control the flow rate of the liquid passing through the plurality of scaffolds 240. The impeller 250 can enable the flowing of the cultivation media through the plurality of openings 231 of each supportive element 242. The impeller 250 can create a high flow rate in low pressure of the liquid passing through the plurality of scaffolds 240. Advantageously, the flow rate of the liquid can be controlled by the impeller 250 to enable the uniform, optimal, and homogeneous distribution of medium parameters (e.g., dissolved oxygen content), as presented hereinabove, along and/or through the surfaces of the plurality of scaffolds 240, thus supporting the cell and/or tissue cultivation thereon.

According to some embodiments, the impeller 250 is configured to circulate the liquid within the bioreactor 210, wherein the liquid flows from the plurality of openings 231 of each supportive element 242, through and/or along the plurality of scaffolds 240 supported by each respective cultivation tray 215 along flow arrow direction 201 (FIG. 3A), through the impeller 250 (by suction) towards the inner supportive element space 243 defined by the plurality of supportive elements 242 via a bottom opening 252 extending through a bottom supportive element 242 (see FIG. 3A), through the plurality of openings 231 of each supportive element 242, and so on. According to further embodiments, the impeller 250 is configured to circulate/mix the liquid within the bioreactor 210 by providing suction to the liquid flowing through and/or along the plurality of scaffolds 240 and delivering it into the inner supportive element space 243 in the flow direction 201.

Advantageously, bioreactor 210 is configured to enable the uniform and/or optimal flow of the liquid between and/or through consecutive scaffolds 240. According to certain embodiments, the liquid is a culture medium that supports the cultivation of cells and/or tissues, cultured on the plurality of scaffolds 240. Moreover, the bioreactor 210 is configured to stir the culture medium such that said culture is homogenously delivered to the cells placed on the scaffolds 240. The design of the bioreactor 210 can allow the efficient seeding of cells onto the scaffolds 240 and their subsequent feeding to create enhanced tissue structures.

According to some embodiments, the system 200 and/or the bioreactor 210 further comprises at least one of a control unit, a pump, medium reservoir, sensor, treatment vessel, dialysis system, incubator, delivery system, cell trap, bubble trap, and combinations thereof, as disclosed herein above in the context of system 100. Each possibility represents a different embodiment.

According to some embodiments, the bioreactor 210 is configured to measure the liquid level therein and/or at least one medium parameter selected from temperature, pH, dissolved oxygen, concentration of one or more nutrients, concentration of one or more waste products, and any combination thereof. According to further embodiments, the bioreactor 210 is further configured to adjust: the liquid level residing therein, the speed rotation of the impeller 250, the at least one medium parameter, or a combination thereof, according to the measurements thereof.

According to some embodiments, bioreactor 210 is a single vessel that is configured to deliver the liquid to the plurality of scaffolds 240 optionally comprising the population of cultured cells and/or tissues as one unit, optionally without utilizing additional appliances (e.g., pump, medium reservoir, etc.), thus enabling the large-scale production of cultured cells, particularly cultured food products at a cost-effective and efficient manner.

According to some embodiments, there is provided a method for producing a plurality of scaffolds 240 comprising cultured cells and/or tissues thereon, comprising the steps of:
a. providing the bioreactor 210 as disclosed hereinabove, comprising a plurality of scaffolds 240 within the plurality of cultivation trays 215;
b. seeding the cells on at least a part of a surface of each scaffold of the plurality of scaffolds 240, utilizing a cell-suspension medium comprising a plurality of cells to be seeded on the scaffolds;
c. circulating a cell culture medium within the bioreactor 210, wherein the cell culture medium is configured to support the cultivation of cells and/or tissues seeded on the scaffolds; and
d. growing the cells to reach at least one pre-set parameter selected from the group consisting of a desired tissue mass; nutrient uptake rate; oxygen uptake rate; waste production rate; and any combination thereof.

According to some embodiments, step (b) comprises flowing the cell-suspension medium into the bioreactor 210, and rotating impeller 250 in order to circulate/mix the cell suspension medium therein. According to further embodiments, the cell suspension medium is circulated/mixed within the bioreactor 210 by flowing from the plurality of openings 231 of each supportive element 242, through and/or along the plurality of scaffolds 240 supported by each respective cultivation tray 215, onwards through the rotating impeller 250, towards the inner supportive element space 243 defined by the plurality of supportive elements 242, and so on. According to still further embodiments, the circulation of the cell-suspension medium within the bioreactor 210 causes the seeding of the cells on the plurality of scaffolds 240.

According to some embodiments, step (b) comprises flowing the cell-suspension medium into the bioreactor 210, wherein the flowing of the cell-suspension medium into the bioreactor 210 causes the seeding of the cells on the plurality of scaffolds 240. According to further embodiments, the cell-suspension medium enters into the bioreactor 210 via at least one liquid port (not shown). According to further embodiments, the cell-suspension medium enters into the bioreactor 210 via a plurality of liquid ports, wherein each liquid port is located at a different section along an inner surface of the bioreactor 210.

According to some embodiments, after the cells were seeded on the plurality of scaffolds 240 in step (b) and prior to step (c), if the cell culture medium is different from the cell-suspension medium, the method further comprises exchanging the cell-suspension medium with the cell culture medium. According to further such embodiments, exchanging the mediums comprises flowing the cell-suspension medium out from the bioreactor 210, and flowing the cell culture medium into the bioreactor 210, optionally via the at least one liquid port. According to alternative embodiments, if the cell culture medium is identical to the cell-suspension medium, the medium is not exchanged.

According to some embodiments, step (c) comprises rotating the impeller 250 in order to circulate/mix the cell culture medium within the bioreactor 210, wherein the cell culture medium flows from the plurality of openings 231 of each supportive element 242, through and/or along the plurality of scaffolds 240 supported by each respective cultivation tray 215, onwards through the impeller 250 towards the inner supportive element space 243 defined by the plurality of supportive elements 242, and so on.

According to some embodiments, the method further comprises a final step of removing at least one scaffold 240 comprising cultured cells and/or tissues from the bioreactor 210.

According to some embodiments, the nutrient is selected from the group consisting of glucose and glutamine. According to some embodiments, the waste product is selected from the group consisting of ammonium and lactate. According to some embodiments, step (b) of seeding the cells comprises seeding cells on one or two of opposite surfaces of each scaffold 240. According to some embodiments, the method is performed under aseptic conditions. According to some embodiments, the cells are non-human-animal cells.

According to some embodiments, the method is configured for producing a cultured food product. According to further embodiments, the cultured food product is cultured meat.

### Scaffolds

As used herein, the term “scaffold” refers to a three-dimensional structure comprising a material that provides a surface suitable for adherence/attachment of cells and the further cultivation (proliferation and/or differentiation and/or maturation) of said cells. A scaffold may further provide mechanical stability and support.

The scaffolds (e.g., scaffolds 140 and/or scaffolds 240) according to certain embodiments of the present invention are porous substrates. In some embodiments, the porous material comprises pores, wherein at least part of the pores is interconnected, optionally forming channels. The inter-connective pore structure and mechanical properties support the cultivation of cells. The porous structure of the scaffold may contribute to allow the cells to penetrate into the depth of the scaffold and allow dispersion to cover it, preferably homogenously. The interconnected pores/channels may further allow a liquid flow into the scaffold and promise the nourishment of the cells.

Any material known in the art to be suitable for forming cell-compatible scaffold, preferably porous, can be used according to the teachings of the present invention, as long as the material can form a three-dimensional structure according to the design of the bioreactors of the invention. According to certain embodiments, the scaffolds of the present invention (e.g., scaffolds 140 and/or scaffolds 240) comprise at least one material selected from the group consisting of polydimethylsiloxane (PDMS), polyester, polypropylene, polylactic acid (PLA), Poly-L-lactic acid (PLLA), poly(lactic-co-glycolic acid) (PLGA), polycaprolactone (PCL), cellulose, silk, hydrogels and combinations and variations thereof. Each possibility represents a separate embodiment of the present invention. According to some embodiments, the hydrogels are natural and synthetic hydrogels, selected from: gelatin, collagen, fibrin, PEG, alginate, chitosan, and other hydrogels known in the art. Each possibility represents a separate embodiment of the present invention. According to certain exemplary embodiments, the hydrogels are edible.

According to certain exemplary embodiments of the present invention, the scaffold is made from an edible material, particularly edible material suitable for human consumption. In some embodiments, the scaffold material comprises protein.

According to certain embodiments, the protein is derived from at least one of a plant, a fungus, an alga, a single cell microorganism and any combination thereof.

According to certain embodiments, the single cell microorganism is selected from the group consisting of yeast, microalgae and bacteria.

According to certain exemplary embodiments, the protein is a plant protein. According to some embodiments, the plant from which the protein is derived is selected from the group consisting of wheat, soybeans, corn, peas, chickpeas, lentils, canola seeds, sunflower seeds, rice, amaranth, lupin, rape-seeds, duckweed, and any combination thereof. Each possibility represents a separate embodiment of the present intention.

According to certain embodiments, the edible scaffold material further comprises at least one edible polysaccharide. According to some embodiments, the polysaccharide is also derived from a plant, fungus, alga, or single cell microorganism, either the same as the protein source or different. Each possibility represents a separate embodiment of the present invention.

In some embodiments, the scaffold comprises at least one plant protein optionally with at least one plant polysaccharide, wherein the plant is selected from the group consisting of wheat, soybean, safflower, corn, peanut, peas, sunflower, chickpea, cotton, coconut, rapeseed, potato and sesame. Each possibility represents a separate embodiment of the present invention.

The proteins and optionally polysaccharides can be obtained from any plant part comprising same, including seeds, leaves, roots, steams, tubers, bulbs and the like, and in some embodiments form part of an extract obtained therefrom. In some embodiments, the extract further comprises additional proteins. In some embodiments, the scaffold comprises pure plant protein.

In some embodiments, the scaffold is of fugal origin. In some embodiments, the scaffold material is obtained from edible fungi, typically macro fungi. Any part of the edible fungi can be used, including the mycelia, hyphae and fruit body (sporocarp).

In some embodiments, the protein or polysaccharide derived from the plant or the fungi or algae comprises a long chain of building blocks. Long chain proteins/polysaccharides provide the scaffold with fibrous texture.

The plant or fungi protein can be texturized to a three-dimensional porous scaffold by any method as is known in the art. Methods of production are described, for example, in WO 2019/016795.

In some embodiments, the scaffold comprises proteins selected from textured protein and a non-textured protein optionally further comprising a polysaccharide.

According to some embodiments, the edible scaffold is a non-extruded product comprising a protein combination comprising at least 40% (w/w) Triticeae gluten out of the total amount of the protein combination and at least one additional type of non-Triticeae protein. According to certain embodiments, the Triticeae plant is a Triticum plant. According to certain exemplary embodiments, the Triticum plant is Triticum aestivum (bread wheat). According to certain additional exemplary embodiments, the Triticum plant is Triticum spelta (Spelt).

According to certain embodiments, the scaffold further comprises water.

According to some embodiments, the at least one additional protein is selected from the group consisting of pea protein, Zea mays protein, soy protein and any combination thereof. Each possibility represents a separate embodiment of the present invention. The initial density of cells seeded on and/or within the scaffold has to be efficient while allowing optimal cell cultivation on/within the scaffold. The number of the cells to be seeded further depends on the porosity of the scaffold material and its liquid absorption capability. The more the scaffold can absorb, the larger the number of cells that can be seeded. In addition, the porosity of the scaffold and the internal organization of scaffold matrix contribute to the retention of the cells within and on the scaffold.

According to certain embodiments of the present invention, cells are seeded on and/or within the plurality of scaffolds before the plurality of scaffolds is placed within the bioreactor. According to certain embodiments, the scaffolds with the seeded cells are placed under conditions allowing cell adherence to the scaffold before the seeded scaffolds are placed in the bioreactor.

According to certain additional or alternative embodiments, the cells are seeded when the plurality of scaffolds is placed within the bioreactor. According to certain exemplary embodiments, the cells are seeded on and/or within scaffolds placed within the bioreactor. According to theses embodiments, bioreactor 210 described hereinabove is typically used.

Before use, the scaffold is typically sterilized. Sterilization may be performed, for example, by gamma-irradiation, by autoclave, by washing with alcohol or by ethylene oxide (EtO) gas treatment.

In some embodiments, the coverage of the scaffold with cells is referred to as “coverage %”. As used herein, “coverage %” denotes the area or volume of the porous scaffold that is in contact with cells throughout the culture process. In some embodiments, the coverage % of the plurality of cells is least 10%, at least 20%, at least 30%, at least 40%, at least 50%, at least 60%, at least 70%, at least 80%, at least 90, or at least 99%. In some embodiments, coverage % of the plurality of cells is 5-20%, 15-30%, 25-40%, 35-50%, 45-60%, 55-70%, 65-80%, 75-90%, 85-100%, or any range therebetween. Each possibility represents a separate embodiment of the present invention.

The seeding and/or the culturing of cells is performed in the presence of a suitable medium. In some embodiment, the medium is a cell culture medium comprises growth factors, small molecules, bioactive agents, nutrients, amino acids, antibiotic compounds, anti-inflammatory compounds, or any combination thereof. Each possibility represents a separate embodiment of the present invention.

In some embodiments, the scaffold is conditioned to enhance adherence of the cells by any current and future methods known in the art.

### Cells

The cell culture bioreactors of the present invention are suitable for cultivating a variety of cells for different purposes. According to certain embodiments, the cells are non-human-animal cells. According to some embodiments, the animal cell are adherent cells.

An exemplary use of the cell culture bioreactors of the invention is for the production of cultured food products, particularly cultured meat. According to these embodiments, the cells are non-human-animal cells. According to some embodiments, the cells are non-genetically modified.

To produce cultured meat, typically two or more types of non-human-animal cells are selected according to the desired type of meat portion to be produced. The produced meat portion can mimic a cut of slaughtered meat, an offal, or designed for the preparation of a certain dish.

According to certain embodiments, the non-human-animal cells comprise stromal and/or endothelial cells and/or fat cells together with at least one cell type according to the desired final meat product, including muscle cells (meat cuts); hepatocytes (liver); cardiomyocytes (heart); renal cells (kidney); lymphoid and epithelial cells (sweetbread made of thymus and pancreas), neural and neuronal cells (brain); ciliated epithelial (tongue), stomach cells (tripe) and progenitor cells thereof. Each possibility represents a separate embodiment of the invention.

According to certain embodiments, the non-human-animal cells are selected from the group consisting of muscle cells, extracellular matrix (ECM)-secreting cells, fat cells, endothelial cells, and progenitors thereof. In some embodiments, non-human-animal cells comprise muscle cells or progenitors thereof and at least one additional type selected from the group consisting of ECM-secreting cells, fat cells, endothelial cells, and progenitors thereof. In some embodiments, the non-human-animal cells comprise muscle cells or progenitors thereof, ECM-secreting cells or progenitors thereof, fat cells or progenitors thereof, and endothelial cells or progenitors thereof.

According to certain embodiments, the non-human-animal is selected from the group consisting of ungulate, poultry, aquatic animals, invertebrate and reptiles. Each possibility represents a separate embodiment of the present invention.

According to certain embodiments, the ungulate is selected from the group consisting of a bovine, an ovine, an equine, a pig, a giraffe, a camel, a deer, a hippopotamus, or a rhinoceros. According to some embodiments the ungulate is a bovine. According to certain exemplary embodiments, the bovine is a cow.

In some embodiments, the non-human-animal derived cells to be seeded on and/or within the scaffold according to the teachings of the present invention comprise pluripotent stem cells. According to certain embodiments, the non-human-animal derived cells to be seeded comprise bovine-derived pluripotent stem cells (bPSCs). According to certain embodiments, the bPSCs are bovine embryonic stem cells. According to certain embodiments, the bPSCs are bovine induced pluripotent stem cells (biPSCs). The seeded bovine-derived adherent cells are grown under conditions enabling differentiation to the desired cell types. In some particular embodiments, the seeded pluripotent bovine-derived cells are differentiated to muscle cells, ECM-secreting cells, fat cells and/or endothelial cells.

In some embodiments, the non-human-animal derived cells to be seeded on the scaffold according to the teachings of the present invention comprise differentiated cells.

In some embodiments, the non-human-animal cells are obtained by differentiating pluripotent stem cells, for example, bovine-derived pluripotent stem cells (PSCs).

The seeding medium and the cultivation medium to be used according to the present invention are those known in the art to be suitable for keeping the viability, and/or proliferation and/or differentiation of the cells.

In some embodiments, the cultivation medium is a serum-free, animal-derived-component-free, liquid medium for non-human-animal cells enriched with a supplement selected from the group consisting of at least one natural colorant, cyanocobalamin (vitamin B12), iron and any combination thereof, wherein the supplement is in amount sufficient to confer red-brown color to the cells.

In some embodiments, the cultivation medium further comprising at least one supplement selected from the group consisting of folate, zinc, selenium, vitamin D, vitamin B, vitamin E, Coenzyme Q10, at least one unsaturated fatty acid, and any combination thereof. Each possibility represents a separate embodiment of the present invention.

In some embodiments, the vitamin D is selected from the group consisting of vitamin D3 and vitamin D2. In some embodiments, the vitamin B is selected from the group consisting of B1, B3, B6 and B12. Each possibility represents a separate embodiment of the present invention.

In some embodiments, the non-saturated fatty acid is selected from the group consisting of Omega 3 fatty acids, Omega 6 fatty acids and a combination thereof.

In some embodiments, the cultivation medium further comprises at least one antimicrobial peptide (AMP) preventing contamination of the cultured cells.

The term “about”, as used herein, when referring to a measurable value such as an amount, a temporal duration, and the like, is meant to encompass variations of +/−10%, more preferably +/−5%, even more preferably +/−1%, and still more preferably +/−0.1% from the specified value, as such variations are appropriate to the disclosed devices, systems and/or methods.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Flow Model

A bioreactor system, comprising a culture vessel bioreactor having a volume of 13.3 liters with a multi-layer scaffold unit (i.e., scaffold unit 120) was designed for flow model analysis. The bioreactor system (i.e., system 100) configuration is as presented in FIG. 1A and the multilayer scaffold unit is as presented FIG. 2A-2B. The multilayer scaffold unit was composed of 12 scaffolds (each of 300×210×3.5 mm) layered one on top of the other with separated supporting elements (grids) (6 mm space between scaffolds). The grids were designed to support and fix a single scaffold layer while keeping a 6 mm gap or space between each neighboring scaffolds. This grid configuration enables free flow of liquid between the different scaffold layers. The multilayer scaffold unit was placed between two manifolds (inlet and outlet), each with twelve pairs of holes, which were adjusted to the twelve gap or space levels between neighboring scaffolds. These manifolds were used to circulate liquid at an adjusted flow rate between the scaffold layers while keeping constant liquid level.

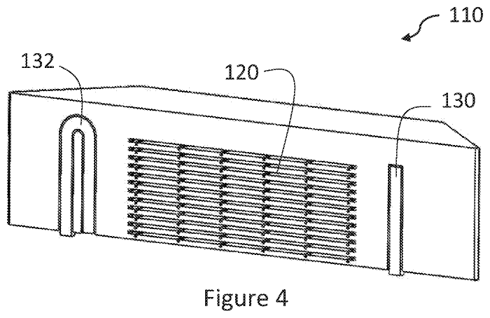

The geometry of the culture vessel bioreactor contains a symmetry plan (as is illustrated in FIG. 4). Therefore, the flow model was developed on half of the vessel geometry. The purpose of the model was to evaluate flow homogeneity, shear stress and oxygen distribution through the scaffold surfaces. Three flow rates were used for flow homogeneity and shear stress evaluation: 0.5, 1 and 1.5 VVD. For oxygen distribution calculation, cell density was taken as 2.67×10⁷ cells/cm² and cell oxygen consumption rate as 0.74 μmol/cell/h. The effect of the liquid level above the upper scaffold was also tested. Two levels were evaluated: 6 mm and 30 mm.

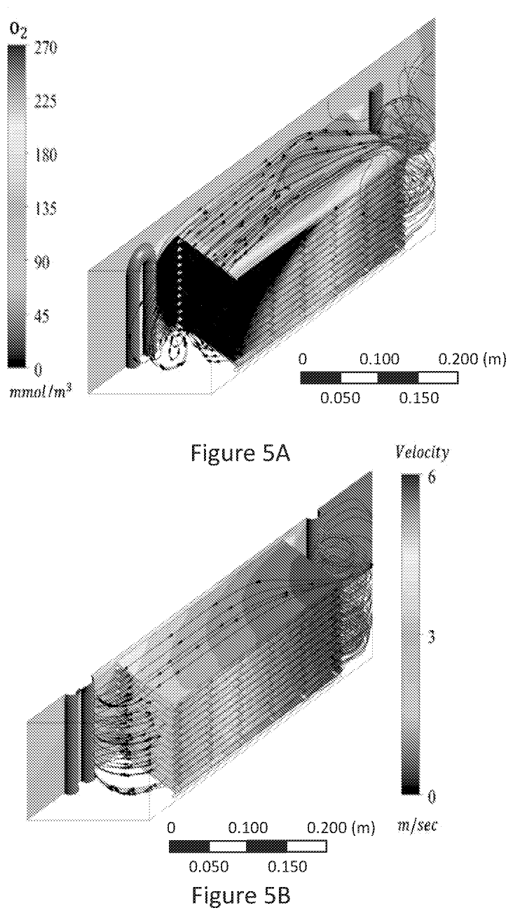

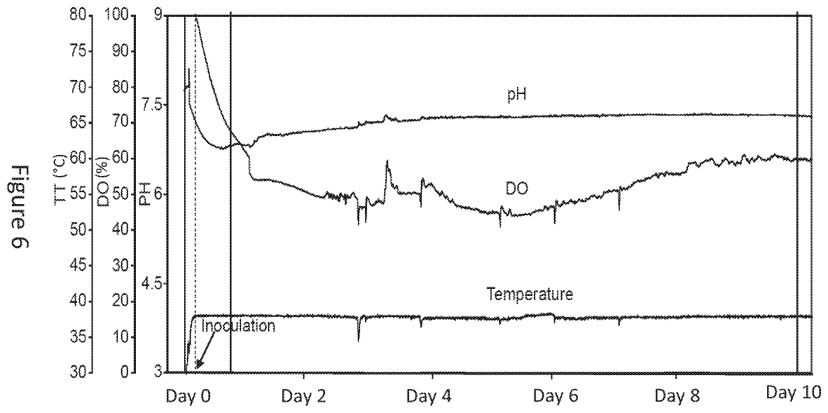

It was found that the level of the liquid above the upper scaffold had an effect on the flow pattern and oxygen distribution. More uniform flow and oxygen distribution between the scaffold layers was obtained when the level above the upper scaffold was 6 mm (FIG. 5B) compared to 30 mm (FIG. 5A). It is contemplated that when the level above the upper scaffold was high, the resistance to liquid flow above this scaffold was lower than between the scaffold layers directing the flow to this location.

A large vortex was developed near the liquid inlet due to the manifold geometry. This vortex had a beneficial effect on the oxygen distribution. The flow rate of the liquid had small effect on the flow field and oxygen distribution patterns. This suggests that the flow rate may be changed without affecting the flow and oxygen distribution. Since the shear stress was relatively low (≤0.2 dyne/cm²) at the highest flow rate, an increase in flow rate can be used to support the oxygen uptake rate of the cells without significantly effecting cell cultivation due to shear stress.

To make sure that oxygen is also supplied to scaffold area in the vicinity of the outlet manifold, a flow circulation switch system was added. This system can be used to alternately switch the direction of the liquid flow. In addition, the oxygen transfer rate can be increased by suppling additional oxygen via headspace while rotating the vessel.

### Example 2: Flow Evaluation

As a proof of concept, a Perspex bioreactor vessel of 0.55 L with four-layer scaffold unit was designed. Four stainless steel grid trays were manufactured to support the four-layer scaffold unit with scaffold dimensions of 115×65×3.5 mm. Two stainless steel manifolds each with 4 pairs of holes were manufactured to support the inlet and outlet flow. The two manifolds were connected to a double head peristaltic pump to support similar inlet and outlet liquid flow rates. The vessel was filled with water at two different levels above the upper scaffold layer. R carotene emulsion was used as a flowing liquid to enable detection of the flow direction by eye. The double head pump was set at 2.3 ml/min and the flow between scaffolds was recorded.

The flow study supported the conclusions of the flow model. Low liquid level was required above the upper scaffold to maintain more homogeneous flow between the scaffold layers. In addition, a large vortex was developed near the liquid inlet as was predicted by the model due to the manifold geometry.

### Example 3: Operation Evaluation

For process evaluation, a stainless-steel culture bioreactor vessel of 0.50 L with a four-layer scaffold unit was designed. Four stainless steel grid trays were manufactured to support the four-layer scaffold unit containing four scaffold layers, with scaffold dimensions of 110×60×3.5 mm. The four scaffolds were seeded with cells and were placed within the culture bioreactor for a cultivation period of 10 days.

Two stainless steel manifolds each with 4 pairs of holes were manufactured to support the inlet and outlet flow. Three peristaltic pumps were used to control the liquid level within the culture bioreactor and to circulate a cell culture medium within the bioreactor system:
1. One peristaltic pump was connected to the inlet manifold. The flow rate of this pump was set to the desired circulation rate.
2. The second peristaltic pump was connected to the outlet manifold. The flow rate of this pump was set at 80% of the desired circulation rate.
3. The third peristaltic pump was connected to a level control and was used as an on/off pump to control the level of the liquid 6 mm above the upper scaffold.

A controlled stirred tank glass bioreactor (1.5 L working volume) was used as a medium reservoir containing the cell culture medium. The pH, DO and temperature of the medium reservoir were controlled at 7.40, 60% air saturation and 38.5° C., respectively. The pH, DO and temperature at the outlet of the culture bioreactor were monitored continuously and the circulation rate was adjusted to maintain the pH level between 7.20 and 7.40, and the DO level between 40-60% air saturation.

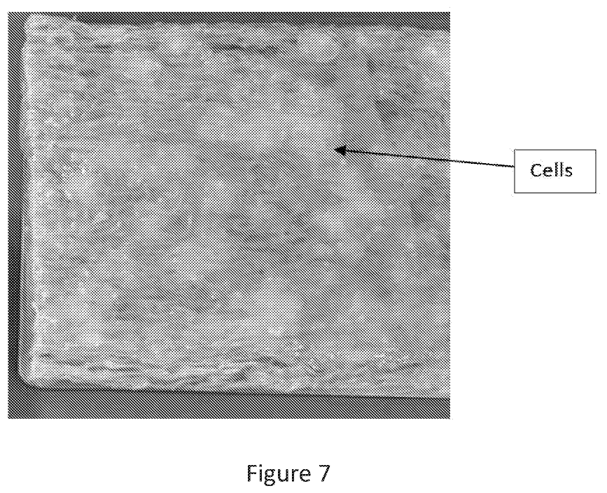

FIG. 6 presents the level of pH, DO and temperature, as they were measured at the outlet of the culture bioreactor during 10 days of cultivation. FIG. 7 presents the cells on the surface of one of the 4 scaffolds at the end of the cultivation period.

From FIGS. 6 and 7 it can be deduced that the process parameters within the culture bioreactor were maintained within the desired operating range, and at the end of the cultivation period, cells were detected on the surface of the scaffolds.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A cultivation system for cell and/or tissue cultivation comprising: at least one cell culture bioreactor comprising:
a first surface comprising an inlet port, a second surface comprising an outlet port, and at least one bioreactor wall, wherein the at least one bioreactor wall is extending from the first surface towards the second surface and is defining an internal chamber configured to accommodate therein at least one scaffold unit;
an inlet manifold fluidly coupled to the inlet port and an outlet manifold fluidly coupled to the outlet port, wherein each manifold comprises openings positioned therealong configured to direct a flow of a liquid; and
at least one scaffold unit comprising a plurality of scaffolds, wherein each scaffold is separated from its neighboring scaffolds to form spaces therebetween enabling liquid flow, wherein at least part of each scaffold comprises cells and/or tissue.

2. The cultivation system of claim 1, further comprising at least one pump, wherein the manifolds are fluidly coupled to the at least one pump, and wherein the at least one pump is configured to control the flow rate of liquid flowing through the at least one scaffold unit and to circulate the liquid within the system.

3. The cultivation system of claim 2, wherein each of the scaffolds comprises at least two surfaces facing opposite directions along and/or in parallel to a longitudinal axis of said scaffold, and wherein none of the surfaces defines an enclosed perimeter across any cross-section thereof.

4. The cultivation system of claim 3, wherein upon operation of the at least one pump, the liquid flows on at least one surface of each scaffold, within the spaces between neighboring scaffolds, or a combination thereof

5. (canceled)

6. The cultivation system of claim 1, wherein the thickness of each of the scaffolds ranges from about 1 mm to about 5 cm.

7-14. (canceled)

15. The cultivation system of claim 1, wherein the scaffold unit further comprises a plurality of supportive elements separating each scaffold from its neighboring scaffolds.

16-17. (canceled)

18. The cultivation system of claim 4, wherein the at least one scaffold unit is disposed within the cell culture bioreactor, so that each of the scaffolds is positioned with the two surfaces being in parallel to a bottom surface of the bioreactor, wherein during the operation of the at least one pump, the liquid passes in parallel to the longitudinal axis within each space along at least one surface of each of the scaffolds, between neighboring scaffolds.

19. The cultivation system of claim 2, wherein the at least one scaffold unit is disposed within the internal chamber of the bioreactor, so that at least one of the openings of each manifold is positioned directly opposing a corresponding space between neighboring scaffolds of the scaffold unit, in order to enable direct liquid flow therethrough.

20. (canceled)

21. The cultivation system of claim 2, comprising a plurality of pumps, wherein a first pump is configured to deliver liquid into the at least one cell culture bioreactor via the inlet manifold at an inlet flow rate, and wherein a second pump is configured to collect liquid from the cell culture bioreactor via the outlet manifold at an outlet flow rate, and, optionally, wherein a third pump is fluidly coupled to the bioreactor, wherein one or more of the first pump, the second pump, and optionally the third pump control the liquid level within the cell culture bioreactor.

22-23. (canceled)

24. The cultivation system of claim 21, wherein the liquid level within the cell culture bioreactor is at least about 0.1 mm above the upper scaffold of the scaffold unit.

25. The cultivation system of claim 1, further comprising at least one sensor, wherein said at least one sensor is configured to measure one or more of: liquid level within the cell culture bioreactor; liquid presence within the cell culture bioreactor; and at least one liquid parameter selected from the group consisting of: temperature, pH, dissolved oxygen content, concentration of one or more nutrients, and concentration of one or more waste products, and optionally, a control unit in operative communication with the at least one sensor, configured to receive measurements of one or more of the liquid level, presence, or the at least one liquid parameter, and adjust it based on the measurement thereof.

26. (canceled)

27. The cultivation system of claim 1, wherein the liquid is a cell culture medium and wherein said cultivation system, further comprising a medium reservoir for supplying cell culture medium into the at least one cell culture bioreactor, wherein the medium reservoir is in fluid communication with the at least one cell culture bioreactor, and wherein said medium reservoir contains a homogenous cell culture medium for the cultivation of cells and/or tissues.

28-31. (canceled)

32. The cultivation system of claim 27, wherein the flow rate is controlled according to a measure of at least one parameter selected from the group consisting of dissolved oxygen, pH, nutrient concentration of one or more nutrients, concentration of one or more waste products, and any combination thereof.

33. The cultivation system of claim 1, wherein the cells are non-human-animal cells, and wherein said system is for use in producing cultured food products.

34. (canceled)

35. The cultivation system of claim 1, wherein the scaffolds are made of at least one edible material.

36. A method for producing at least one scaffold unit comprising a plurality of scaffolds comprising cultured cells and/or tissues, the method comprising the steps of:
a. providing at least one scaffold unit comprising a plurality of scaffolds, wherein each scaffold is separated from its neighboring scaffolds to form spaces therebetween enabling liquid flow, wherein each scaffold is porous;
b. seeding cells on at least part of a surface of each scaffold of the plurality of scaffolds or within each scaffold of the plurality of scaffolds;
c. optionally placing the scaffold unit under conditions enabling adherence of the seeded cells thereto, until said cells are at least partially covering at least one surface of each of the scaffolds, or until the cells are covering at least a part of the pores and/or channels of each of the scaffolds;
d. placing the scaffold unit in a cell culture bioreactor comprising an inlet manifold and an outlet manifold, wherein each manifold comprises a plurality of openings configured to direct a flow of a liquid therethrough, wherein the manifolds are connected to at least one pump configured to control the flow rate of said liquid;
e. operating the at least one pump to deliver the liquid from the inlet manifold, along said spaces between neighboring scaffolds of at least one scaffold unit, and into the outlet manifold; and
f. cultivating the cells on and/or within the plurality of scaffolds,
thereby producing a plurality of scaffolds comprising cultured cells and/or tissues.

37. The method of claim 36, wherein step (b) of seeding the cells comprises administering the cells directly into each scaffold, or into the spaces separating neighboring scaffolds; step (e) comprises delivering the cell culture medium (i) from the plurality of openings of the inlet manifold into each space separating neighboring scaffolds of the scaffold unit, (ii) from the spaces into the plurality of openings of the outlet manifold, and (iii) from the plurality of openings of the outlet manifold into the plurality of openings of the inlet manifold, thereby circulating the liquid within the bioreactor; and step (f) comprises cultivating the cells on and/or within the plurality of scaffolds to reach at least one pre-set parameter selected from the group consisting of a desired tissue mass; nutrient uptake rate; oxygen uptake rate; waste production rate; and any combination thereof, thereby producing a plurality of scaffolds comprising cultured cells and/or tissues thereon and/or therewithin.

38-39. (canceled)

40. The method of claim 36, wherein the at least one pump comprises a plurality of pumps, wherein during step (e) a first pump is delivering the liquid into the cell culture bioreactor via the inlet manifold at an inlet flow rate, wherein a second pump is collecting used liquid from the cell culture bioreactor via the outlet manifold at an outlet flow rate, and optionally wherein a third pump is fluidly coupled to the bioreactor, wherein one or more of the first pump, the second pump, and optionally the third pump control the liquid level within the cell culture bioreactor.

41. (canceled)

42. The method of claim 36, wherein the liquid is a cell culture medium, the cells are non-human-animal cells and the scaffold unit is edible.

43-47. (canceled)

48. A scaffold unit comprising a plurality of scaffolds comprising cultured cells and/or tissues thereon and/or therewithin, produced by the method of claim 36.

49. A cultured food product comprising at least one scaffold unit according to claim 48, or a portion thereof.

50. A bioreactor configured to deliver a liquid to a plurality of scaffolds, the bioreactor comprising:
(a) at least one bioreactor wall defining an internal chamber and is extending between a bottom surface and an upper surface, in parallel to a vertical axis;
(b) at least one main tube, positioned along the vertical axis, and is extending perpendicularly from the upper surface;
(c) a plurality of cultivation trays disposed within the internal chamber, wherein said plurality of cultivation trays extends radially from at least one supportive element directly or indirectly attached to the main tube, wherein each cultivation tray is configured to support at least one scaffold and is further configured to enable a liquid to flow along and/or through each scaffold; and
(d) at least one mixing apparatus attached to the main tube, wherein the mixing apparatus is configured to circulate the liquid within the bioreactor,
wherein the bioreactor is configured to deliver the liquid to a plurality of scaffolds disposed therein.

51. The bioreactor according to claim 50, wherein the at least one supportive element is shaped as a hollow tube encompassing the main tube, thus defining an inner supportive element space therebetween which is configured to enable the liquid to flow therethrough, and wherein the at least one supportive element comprises a plurality of openings spaced apart from each other along a circumference thereof.

52. The bioreactor according to claim 51, wherein a portion of the plurality of openings is located above each respective cultivation tray, and wherein each cultivation tray comprises at least one scaffold disposed therein or thereon.

53. The bioreactor according to claim 52, wherein the plurality of cultivation trays extends radially from a corresponding plurality of supportive elements fluidly coupled to one another, wherein each supportive element is attached to at least one cultivation tray.
